(19) United States
(12) Patent Application Publication
(10) Pub. No.: US 2006/0159617 A1
(43) Pub. Date: Jul. 20, 2006

(54) Small technetium-99m and rhenium labeled agents and methods for imaging tissues, organs and tumors

(76) Inventors: Ashfaq Mahmood, Newton Center, MA (US); Zhen Cheng, Mountain View, CA (US); Alun G. Jones, Newton Center, MA (US); Alan Davison, North Falmouth, MA (US)

Correspondence Address: EDWARDS & ANGELL, LLP, P.O. BOX 55874, BOSTON, MA, 02205, US

(21) Appl. No.: 10/534,225

(22) PCT Filed: Nov. 8, 2003
(86) PCT No.: PCT/US03/35618

§ 371 Date: Mar. 17, 2006

Related U.S. Application Data
(60) Provisional application No. 60/424,980, filed on Nov. 8, 2002.

(51) Int. Cl. A61K 51/00; C07F 13/00; C07F 5/00; C07D 211/26

(52) U.S. Cl. 424/1.11; 534/11; 534/14; 546/229

(57) ABSTRACT

The present invention relates to compounds and related technetium and rhenium complexes thereof which are suitable for imaging or therapeutic treatment of tissues, organs, or tumors. In another embodiment, the invention relates the methods of imaging tissues, organs or tumors using radiolabeled metal complexes, particularly tissues, organs, or tumors which express certain receptors to which the compounds or complexes of the invention have an affinity. The present invention also relates to methods of treating cancer, particularly those cancer lines which express certain receptors to which the compounds or complexes of the invention have an affinity. In yet another embodiment, the present invention provides methods of imaging and/or inhibiting receptors or neuroreceptors using compounds or complexes of the invention which have an affinity for the receptor or neuroreceptor to be imaged and/or inhibited.

This application claims the benefit of U.S. Provisional Patent Application 60/424,980, filed Nov. 8, 2002, which application is incorporated herein by reference.

### STATEMENT REGARDING FEDERALLY SPONSORED RESEARCH OR DEVELOPMENT

This invention was supported by National Institute of Health (NIH) Grant No. R37 CA34970. The United States government has certain rights to the invention.

### FIELD OF THE INVENTION

The present invention relates to small molecular radiometal diagnostic agents for imaging tissues, particularly tissues expressing or overexpressing one or more receptors for which the diagnostic agents of the invention have an affinity. More specifically, the present invention relates to small molecular diagnostic agents for imaging tissues, which include tumors, various brain tissues, and other organs and diseased states, bearing certain preferred receptors and corresponding therapeutic complexes for treating the same. Preferred agents of the invention includes technetium and rhenium complexes having a tertiary amine pharamacophore linked to a chelating ligand. Typically preferred technetium and rhenium complexes of the invention include those comprising a disubstituted piperidine group or a tertiary amino group, which is substituted with at least one carbocyclic or heterocyclic substituted alkyl group.

### BACKGROUND OF THE INVENTION

Signal transduction in cells is defined as a biochemical communication from one part of the cell to another. Such communication between and within cells is carried out by, for example, binding of an extracellular ligand to a specific cell surface transmembranal receptor which are coupled to G-proteins in the cytoplasm or by regulation of ion channels such as Ca²⁺, Na⁺, K⁺, Cl⁻, or the like. Binding of the ligand to receptor induces a transmembranal signal which results in activation (or deactivation) of various cellular processes and functions. Small synthetic molecules that target these cellular receptors at the cell surface or intracellularly, with a high degree of specificity are highly desirable because of their rapid and increased tissue penetration, reduced immunogenicity and reduced metabolism when compared to monoclonal antibodies, their fragments or polypeptides.

The use of small molecules with gamma or positron emitting radiolabels also provides a means for non-invasive visualization and imaging of targeted receptors in both normal and diseased states. This has led to a search for small molecules labeled with positron emitting isotopes and single photon emitting isotopes that target various receptors and permit the non-invasive visualization of these receptors in the targeted tissues. See, for example, Nuclear Medicine Biology Vol 24, 485-498 1997. See also John (U.S. Pat. No. 5,919,934), Nuclear medicine Biology vol 28, 657-666 2001, and international publications to Mach (WO/0180905A2and WO00/71171 A2).

Serotonin also known as 5-hydroxytryptamine (5-HT) is an important neurotransmitter molecule and various receptor subtypes have been identified, among these receptor subtypes 5HT_{1A} is one of the best characterized and studied as it is implicated in anxiety, depression, hallucinogenic behavior as well as in dementia such as Alzheimer's desease. See, for example, Neuropharmacology vol 38, 1083-1152 1999 and Euro. Journal of Nucl. Med. Vol 28, 113-129, 2001. A number of ^{99m}Tc-complexes with 2-methoxyphenylpiperazines have been investigated for binding and visualization of the 5HT_{1A} receptor, however no aryl-piperdine linked technetium or rhenium-complexes have been investigated or reported for this purpose. See, also Nuclear Medicine Biology Vol 24, 485-498 1997; Technetium, rhenium and other metals in chemistry and nuclear medicine 5. Padova: Servizi Grafici Editoriali, 1999:393-9; and European Journal of Nuclear Medicine vol 29(2) 263-275, 2002.

In addition to its role as a neurotransmitter, 5HT can also function as a growth factor and is found in most neuroendocrine cells of the human prostate and in human prostate cancer cell lines. Several articles have reviewed 5HT's role in prostate cancer cell lines, for example, Anticancer Res 1987;7:1-12; Cancer Res 1991;51:2498-2505; and Cancer 1992;70:254-68. A 5HT_{1A} receptor antagonist has also been shown to inhibit prostate tumor cell growth in vivo (Anticancer Res 1994; 14:1215-20).

Sigma-receptors are recognized to be intra-cellular cytoplasmatic sites, distinguished in at least σ-1 and σ-2 subtypes (with a σ-3 site also postuated). Both subtypes are widely distributed in CNS (central nervous system), liver, kidney, lung, and in endocrine, immune, and reproductive tissues, and are overexpressed in several tumor cell lines (Vilner et al Cancer Res. 1995, 55, 408-413.). A recent review recites several potential applications for compounds having affinity for sigma receptors. Moreover, preliminary studies indicate that certain sigma agonists or sigma antagonists may be suitable for imaging or treating various cancers. See, for example, Wayne Bowen and Fabian Moebius (Pharm. Acta Helv. 2000, 74, 211-218; Trends Pharmacol. Sci. 1997, 18,67-70.).

Similar to the serotonin receptors, the sigma receptors (including sigma-1 and sigma-2) that are normally expressed in the brain are also over expressed in a number of tumors. Sigma receptors, originally thought be a subclass of opiate receptors, are nondopaminergic, nonopiate membrane proteins that possess high affinity for haloperidol and various other neuroleptics. Two subtypes, termed σ-1 and σ-2 have now been identified.

The (+)-benzomorphans ((+)-[³H]-pentazocine) selectively label the σ-1 sites; the enantiomeric (−)-benzomorphans show lower affinity and no differentiation between the two, sites. The σ-2 sites, however are identified with [³H]-DTG a nonselective s-1/s-2 ligand in the presence of dextrallophan, which masks binding of the σ-1 sites (Pharmacological reviews vol 42(4), 355-402, 1990).

Several studies have now been reported on the overexpression of sigma receptors in human and murine tumors including human melanoma, small cell lung carcinoma, human breast carcinomas and both androgen-dependent and -independent prostate carcinomas (Cancer Research vol 55(2), 408-413, 1995; Bioconjugate Chem 1997;8:304-9; and Nucl Med Biol 1998;25:189-94). See also John (U.S. Pat. No. 5,919, 934), Nuclear medicine Biology vol 28, 657-666 2001, and international publications to Mach (WO/0180905 A2 and WO 00/71171 A2).

Adrenoreceptors, including α₁ receptors, are another family of G-protein-coupled receptors expressed in the brain, and are expressed in prostatic deseases such as benign prostatic hyperplasia (BPH) and are used for the treatment of this desease (Journal of Andrology vol 12, 389-394, 1991 and Jour. Medicinal Chem. Vol 40, 1293-1315, 1997).

The wide spread availability of ^{99m}Tc in most major hospitals and the routine use and practicality of SPECT imaging in nuclear medicine gives impetus to the development of such receptor-imaging agents labeled with technetium-99m. The use therapeutic rhenium-186 or rhenium-188 may permit the radiotherapy of diseases to which these small receptor-specific complexes bind.

The most widely used isotope in clinical nuclear medicine, technetium-99m, possesses ideal characteristics (t_{1/2}=6.02 h, 140 keV monoenergeric γ-emission) for nuclear medicine imaging and is available on demand from a ⁹⁹Mo-^{99m}Tc generator system.

Thus, new and useful radiolabeled diagnostic agents, including ^{99m}Tc and ¹⁸⁶Re and ¹⁸⁸Re labeled diagnostic agents, for imaging tissues, particularly tissues expressing or over expressing one or more of the receptors discussed supra, would be desirable. Moreover ^{99m}Tc and ¹⁸⁶Re and ¹⁸⁸Re labeled diagnostic and therapeutic agents suitable for use in imaging or treating melanoma, prostate cancer, other tumor, or diseased states, various portions of the brain or other tissues expressing or overexpressing one or more receptors discussed supra would be desirable.

### SUMMARY OF THE INVENTION

The present invention provides new radiolabeled diagnostic and therapeutic agents which comprise a metal or radiometal center. Preferred radiometals include 99m-technetium and one or more radioactive and non-radioactive isotopes of rhenium. Preferred agents are useful for in-vivo and in-vitro imaging of tumors, such as neoplasms, carcinoma and melanoma, or tissues or organs expressing one or more proteins, receptors or neuroreceptors, such as serotonin receptors, α receptors, σ receptors, calcium channel receptors or emopamil binding proteins adrenergic receptors, adrenoceptors receptors, dopamine receptors, and any subclass of receptors or proteins thereof. Particularly preferred agents are useful for in-vivo and in-vitro imaging. Preferred agents of the present invention comprise an oxotechnetium core (Tc═O) or an oxorhenium core (Re═O) linked to a tertiary amine pharnacophore such as, but not limited to, a N-substituted piperidine pharmacophore.

Thus, compounds provided by the invention include those according to Formula I: 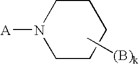
wherein

A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;

B is independently selected at each occurrence of B from the group consisting of hydrogen, optionally substituted alkyl, optionally substituted alkenyl, optionally substituted alkynyl, optionally substituted alkoxy, halogen, hydroxy, optionally substituted alkoxyalkyl, optionally substituted amino, optionally substituted mono and dialkyl amino, optionally substituted aryl, optionally substituted aralkyl, optionally substituted cycloalkyl, optionally substituted heteroalicyclic, optionally substituted heteroaralkyl, optionally substituted heteroaryl, and —X—Y, wherein at least on occurrence of B is not hydrogen;

X is a linking group comprising a backbone chain having 1 to about 8 atoms, the backbone chain can optionally include ester, amide, ether or thioether linkages in the backbone chain;

k is an integer from about 1 to about 3; and

Y is a group capable of chelating to at least one metal ion,

wherein at least one of A or B is chosen to be -X-Y.

Preferred compounds of Formula I provided by the present invention include those compounds having one B group, e.g., k=1, attached at the 2, 3, or 4 position of the piperidine ring. Other preferred compounds of Formula I provided by the present invention have two B groups, e.g., k=2, where both B groups are attached together or independently at the two (ortho), three (meta) or four (para) position of the piperidine ring.

Other compounds provided by the invention include those according to Formula II:
Y—X—NR₁R₂ II
where Y is a chelating ligand capable of binding a metal ion, X is a linking group containing a backbone chain having 1 to about 8 atoms, and R₁ and R₂ each are independently selected unsubstituted alkyl groups having from 1 to about 8 carbon atoms and substituted alkyl groups having from 1 to about 8 carbon atoms which are substituted with one or more groups selected from optionally substituted aryl, preferably having from 6 to 12 carbon atoms, optionally substituted aralkyl, preferably having from 7 to 18 carbon atoms, optionally substituted cycloalkyl, preferably having from 3 to 8 carbon atoms, optionally substituted heteroalicyclic, preferably having from 3 to 8 carbon atoms and between 1 and 3 heteroatoms in the heteroalicyclic ring, optionally substituted heteroaralkyl, preferably having from 7 to 18 carbon atoms and between 1 and 3 heteroatoms in the heteroaryl ring, optionally substituted heteroaryl, preferably having from 7 to 18 carbon atoms and between 1 and 3 heteroatoms in the heteroaryl ring, wherein at least one of R₁ or R₂ is a substituted alkyl group.

Preferred compounds of Formula II include those compounds in which X is a C₂₋₈-alkylene, R₁ is an optionally substituted C₁₋₆alkyl group and R₂ is an optionally substituted (aryl)C₁₋₄alkyl group or an optionally substituted (heteroaryl)C₁₋₄alkyl group.

Preferred linking groups, X, are lower alkyl groups having from 1 to about 8 atoms in the backbone such as, e.g., —(CH₂)ₙ—, ether groups having 1 to 8 atoms in the backbone such as, e.g., —(CH₂)ₙ—O—(CH₂)ₘ—, ester groups having 1 to 8 atoms in the backbone such as, e.g., —(CH₂)ₙ—CO—O—(CH₂)ₘ—, thioether groups having 1 to 8 atoms in the backbone such as, e.g., —(CH₂)ₙ—S—(CH₂)ₘ—, and amido groups having 3-8 atoms in the backbone such as, e.g., —(CH₂)ₙCO—NH—CH₂CH₂— or —(CH₂)ₙCO—NH—, where n and m are non-negative integers and the sum n+m is typically between about 1 and about 8. Particularly preferred linking groups X have between about 2 and about 5 atoms in the backbone.

Linking groups X may optionally have one or more substituents attached to the backbone chain including pendant aromatic groups. Preferred substituents include alkyl groups having from 1 to about 6 carbon atoms and from 0 to about 3 oxygen, sulfur, or oxidized sulfur atoms, hydroxyl, amino, carboxyl, alkoxy groups having from 1 to about 6 carbon atoms, aminoalkyl groups having from 1 to about 6 carbon atoms, dialkylaminoalkyl groups where each alkyl group has from about 1 to about 6 carbon atoms, halogen atoms including F, Cl, Br, and I, aromatic groups having about 5 to about 18 ring atoms which may include 0, 1, 2, or 3 N, O or S ring atoms.

The compounds of the invention are then complexed with a metal ion using methods well known in the art to provide metal complexes. Imaging applications typically comprise metal complexes which are radiolabelled and more typically comprise at least one radiolabelled metal ion (e.g., a radioactive metal ion). Therapeutic applications typically comprise metal complexes of the invention which are cytotoxic and may comprise cold (e.g., non-radioactive metal ions) or radiolabelled metal ions or a combination thereof. Typical radiolabeled complexes of the invention are cationic or neutral. Preferred radiometal ions include isotopes of metal ions that emit α, β⁻, β⁺ or γ radiation, including metal ions selected from the group consisiting of technetium, rhenium, yttrium, copper, gallium, indium, bismuth, platinum and rhodium. Particularly preferred radiolabeled complexes of the invention comprise a technetium or rhenium metal ion.

The present invention further provides methods for in-vivo or in-vitro imaging of at least one tissue expressing one or more protein or receptor for which radiolabeled complexes have affinity, the method comprising the steps of

providing a radiolabeled complex comprising a metal ion and a compound according to Formula I, Formula II or any subformula thereof;

contacting the tumor(s) with the radiolabeled complex; and

making a radiographic image to image the tissue(s).

Preferred tissues suitable for use in the imaging methods of the present invention are not particularly limited. However, typically preferred tissues include those tissue which express or over-express one or more proteins, receptors or neuroreceptors, such as serotonin receptors, α receptors, σ receptors, calcium channel receptors or emopamil binding proteins adrenergic receptors, adrenoceptors receptors, dopamine receptors, and any subclass of receptors or proteins thereof. Preferred tissues which can be imaged by the methods of the invention include brain tissue, organs, tumors and cells or tissues and the like which express such proteins and/or receptors.

The present invention also provides methods for in-vivo or in-vitro imaging of at least one tumor comprising the steps of:

providing a radiolabeled complex comprising a metal ion and a compound according to Formula I, Formula II or any subformula thereof;

contacting the tumor(s) with the radiolabeled complex; and

making a radiographic image to image and/or visualize the tumor(s).

In preferred embodiments, the radiolabeled complexes are injected into a mammal to obtain an image of at least one tissue, organ, or tumor. Preferable radiolabeled complexes accumulate in the tissue, organ, or tumor. Images are obtained by conventional techniques such as use of a radioscintillation camera such as those used for positron emission tomography (PET), single photon emission tomography (SPECT) or the like.

The present invention also provides methods for the treatment of cancer or disease comprising the steps of:

providing a cytotoxic metal complex comprising a metal ion and a compound according to Formula I or II or any subformula thereof; and

contacting the tumor(s) or tissue(s) with the cytotoxic metal complex.

### BRIEF DESCRIPTION OF THE DRAWINGS

FIG. 1 is a plot of σ₁ binding affinity for various Re complexes of the invention compared to [³H]-Pentazocine;

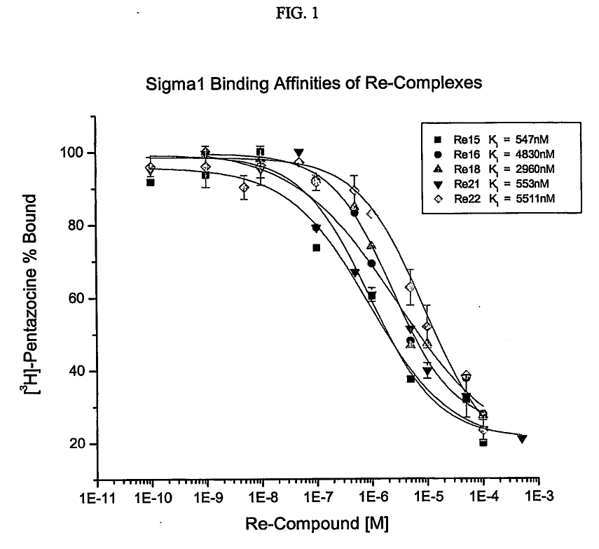

FIG. 2 is a plot of σ₂ binding affinity for various Re complexes of the invention compared to [³H-DTG];

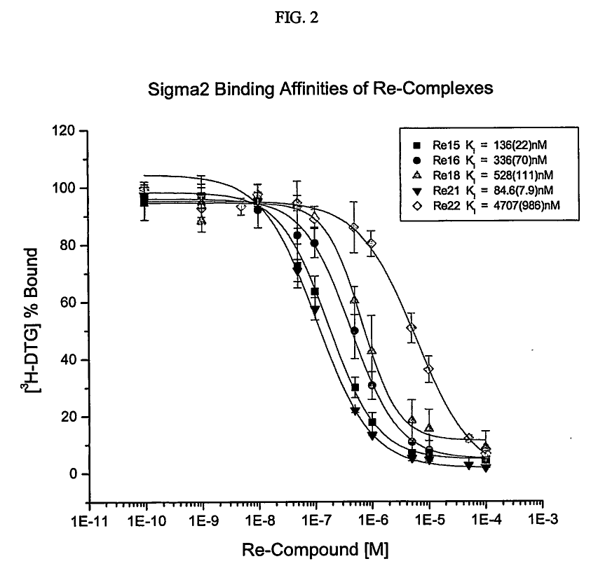

FIG. 3 is a plot of α₁ binding affinity of various Re complexes of the invention compared to [³H]-prazosin;

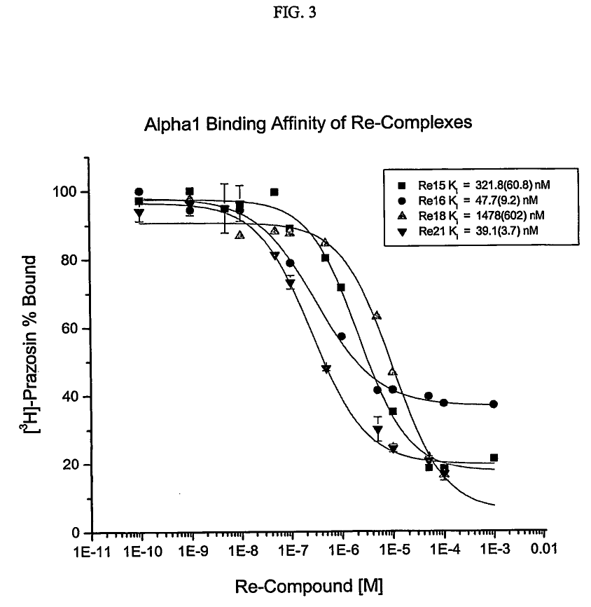

FIG. 4 is a plot of 5HT_{1A} binding affinity of various Re complexes of the invention compared to [3H-8-OH-DAPT];

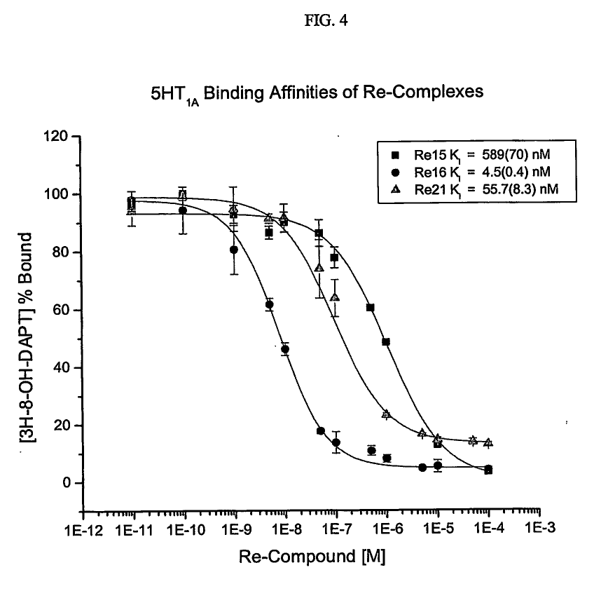

FIG. 5 is an ORTEP representation of complex Re-24 determined by X-ray crystallography;

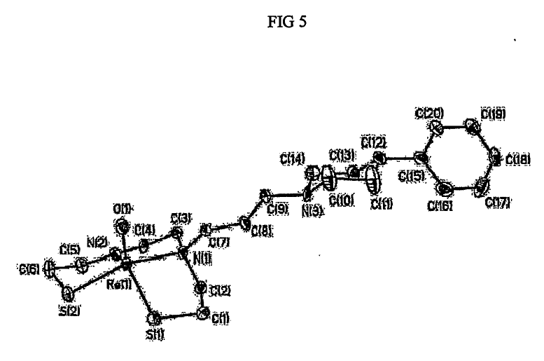

FIG. 6 is a plot of σ₁ binding affinity for various Re complexes of the invention compared to [³H]-Pentazocine;

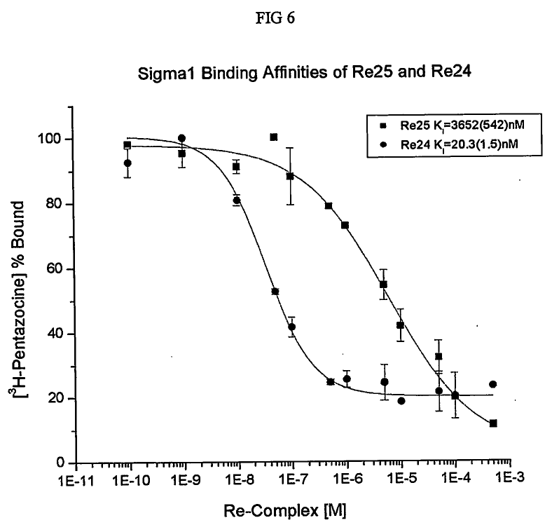

FIG. 7 is a plot of σ₂ binding affinity for various Re complexes of the invention compared to [³H-DTG];

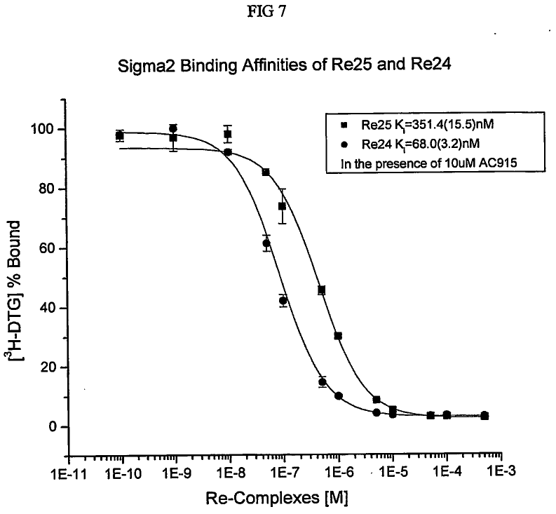

FIG. 8 is a plot of α₁ binding affinity of various Re complexes of the invention compared to [³H]-prazosin;

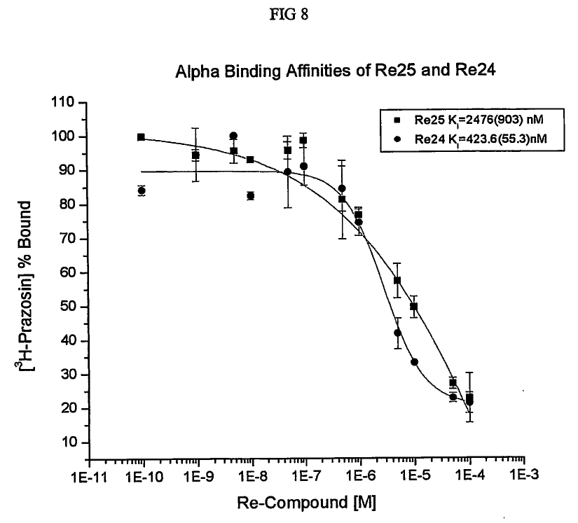

FIG. 9 is a plot of 5HT_{1A} binding affinity of various Re complexes of the invention compared to [3H-8-OH-DAPT];

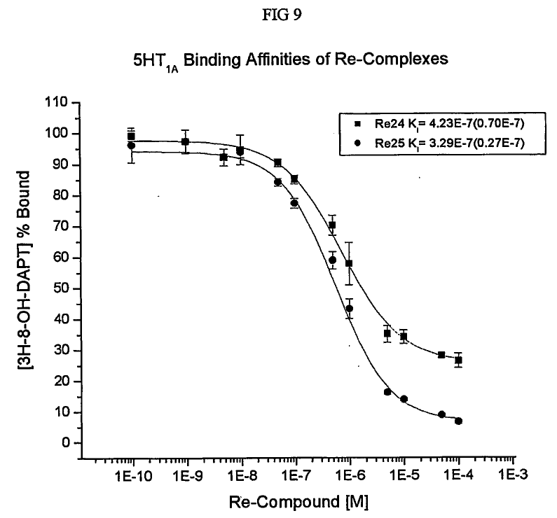

FIG. 10 is a plot of σ₁ binding affinity for various Re complexes of the invention compared to [³H]-Pentazocine;

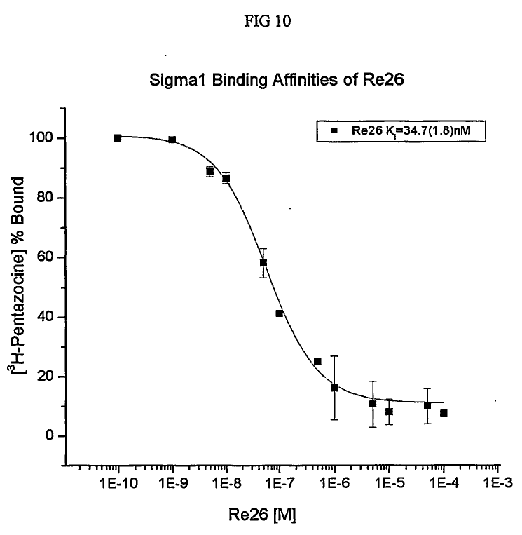

FIG. 11 is a plot of σ₂ binding affinity for various Re complexes of the invention compared to [³H-DTG];

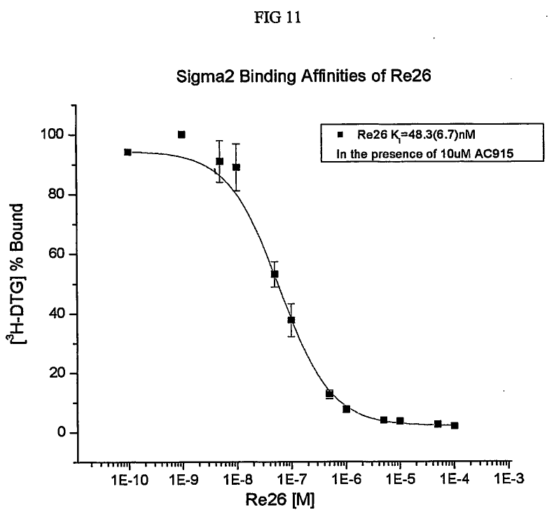

FIG. 12 is a plot of α₁ binding affinity of various Re complexes of the invention compared to [³H]-prazosin; and

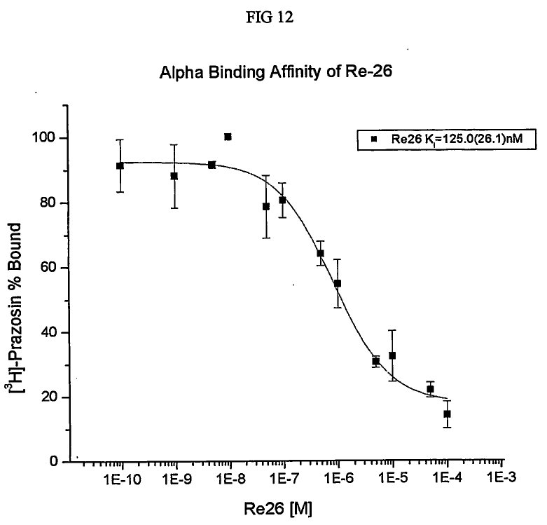

FIG. 13 is a plot of 5HT_{1A} binding affinity of various Re complexes of the invention compared to [3H-8-OH-DAPT].

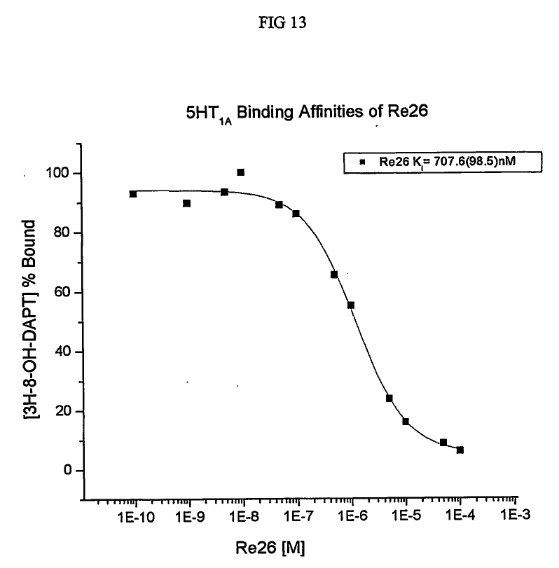

### DEFINITIONS

Tr and Trt refer to trityl groups, e.g., triphenylmethyl groups.
DTG refers to ditolyl guanidine.
AADT refers to amino-amido-dithiolate ligands, preferred AADT ligands have a N-[2-(2-mercapto-ethylamino)-ethylamino]-ethanethiol structure.
DADT refers to diamino-dithiolate ligands, preferred DADT ligands have a 2-[2-(2-mercapto-ethylamino)-ethylamino]-ethanethiol structure.

The term “substituted”, as used herein, means that any one or more hydrogens on the designated atom is replaced with a group selected from the defined list, provided that the designated atom's normal valence is not exceeded, and that the substitution results in a stable compound. When a substituent is keto (i.e., =0), then 2 hydrogens on the atom are replaced. Keto substituents are not directly attached to aromatic ring atoms.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible provided that such combinations result in stable compounds.

As indicated herein, various substituents of the compounds of the present invention and various formulae set forth herein are “optionally substituted”, including, e.g., a linker or carboxylate leaving group. When substituted, those substituents can be substituted at one or more of any of the available positions, typically 1, 2, 3, 4, or 5 positions, by one or more suitable groups such as those disclosed herein.

Suitable groups or “substituted” moieties for hydrogen atoms in compounds of the invention include, e.g., halogen such as fluoro, chloro, bromo or iodo; cyano; hydroxyl; nitro; azido; alkanoyl, such as a C₁₋₆ alkanoyl group such as acyl and the like; carboxamido; alkyl groups including those groups having 1 to about 12 carbon atoms, preferably 1-6 carbon atoms; alkenyl and alkynyl groups including groups having one or more unsaturated linkages and from 2 to about 12 carbon atoms, preferably 2-6 carbon atoms; alkoxy groups including those having one or more oxygen linkages and from 1 to about 12 carbon atoms, preferably 1-6 carbon atoms; aryloxy groups such as phenoxy and benzyloxy; alkylthio groups including those moieties having one or more thioether linkages and from 1 to about 12 carbon atoms, preferably 1-6 carbon atoms; alkylsulfinyl groups including those moieties having one or more sulfinyl linkages and from 1 to about 12 carbon atoms, preferably 1-6 carbon atoms; alkylsulfonyl groups including those moieties having one or more sulfonyl linkages and from 1 to about 12 carbon atoms, preferably 1-6 carbon atoms; aminoalkyl groups such as groups having one or more N atoms and from 1 to about 12 carbon atoms, preferably 1-6 carbon atoms; carbocyclic aryl groups having 6 or more carbons, particularly phenyl and benzyl (e.g., wherein an Ar group can be substituted or unsubstituted biphenyl moiety); arylalkyl having 1 to 3 separate or fused rings and from 6 to about 18 carbon ring atoms, with benzyl being a preferred group; arylalkoxy having 1 to 3 separate or fused rings and from 6 to about 18 carbon ring atoms, with O-benzyl being a preferred group; or a heteroaromatic or heteroalicyclic group having 1 to 3 separate or fused rings with 3 to about 8 members per ring and one or more N, O or S atoms.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are lower alkyl groups having from 1 to about 6 carbon atoms. The term C₁₋₆ alkyl as used herein means alkyl groups consisting of 1 to 6 carbon atoms, which may contain a cyclopropyl moiety.

“Cycloalkyl” is intended to include saturated ring groups, having a specified number of carbon atoms, such as cyclopropyl, cyclobutyl, cyclopentyl, or cyclohexyl and bridged or caged saturated ring groups such as norbornane or adamantane and the like. Preferred cycloalkyl groups are cycloalkyl groups having from 3 to about 8 ring atoms. The term C₃₋₈ cycloalkyl as used herein means cycloalkyl groups consisting of a aliphatic ring with 3 to 8 atoms in the ring.

“Alkenyl” is intended to include hydrocarbon chains of either a straight or branched configuration comprising one or more unsaturated carbon-carbon bonds, which may occur in any stable point along the chain such as, e.g., ethenyl and propenyl. Preferred alkenyl groups are lower alkenyl groups having from 2 to about 6 carbon atoms. The termi C₂₋₆ alkenyl as used herein means alkenyl groups consisting of 2 to 6 carbon atoms.

“Alkynyl” is intended to include hydrocarbon chains of either a straight or branched configuration comprising one or more triple carbon-carbon bonds that may occur in any stable point along the chain such as, e.g., ethynyl and propynyl. Preferred alkynyl groups are lower alkynyl groups having from 2 to about 6 carbon atoms. The term C₂₋₆ alkynyl as used herein means alkynyl groups consisting of 2 to 6 carbon atoms.

As used herein, the term “heterocyclic group” is intended to include saturated, partially unsaturated, or unsaturated (aromatic) groups having 1 to 3 (preferably fused or spiro) rings with 3 to about 8 members per ring at least one ring containing an atom selected from N, O or S. The nitrogen and sulfur heteroatoms may optionally be oxidized. The term “heteroalicyclic” or “heterocycloalkyl” is used to refer to saturated or partially unsaturated heterocyclic groups.

As used herein, the term “aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

“Haloalkyl” is intended to include both branched and straight-chain saturated aliphatic hydrocarbon groups having the specified number of carbon atoms, substituted with 1 or more halogen (for example —Cᵥ(Xⁱ)_{wi}(H_{2v+1-Σ(wi)}) where v=1 to 6; Xⁱ=F(i=1), Cl(i=2), Br(i=3), I(i=4) and Σw_{I}≦2v+1). Examples of haloalkyl include, but are not limited to, trifluoromethyl, trichloromethyl, pentafluoroethyl, and pentachloroethyl. Preferred haloalkyl groups are lower halolkyl groups having from 1 to about 6 carbon atoms. The term C₁₋₆ haloalkyl as used herein means haloalkyl groups consisting of 1 to 6 carbon atoms.

As used herein, the term “hydrocarbon group” is intended to include alkyl, cycloalkyl, alkenyl, alkynyl, and aryl groups or a group that comprises a combination of two or more alkyl, cycloalkyl, alkenyl, alkynyl or aryl group regions. Hydrocarbon groups may further comprise heteroatoms such as N, O, F, Si, S, Cl, Br and the like. Preferably, hydrocarbon groups have from 0 to about 3 heteroatoms. The term lower hydrocarbon group as used herein means a hydrocarbon group consisting of 1 to 6 carbon atoms which may include 1, 2, or 3 heteroatoms.

As used herein, the term “lipophilic group” refers to any hydrophobic group that is soluble in or miscible with lipids, hydrocarbons and other hydrophobic materials. Examples of lipophilic groups include, but are not limited to, long-chain C₆-C₃₂ alkyl groups that include linear alkyls, branched alkyls with one or more branch points or linear or branched alkyls which include one or more C₃-C₈ cycloalkane groups, long-chain C₆-C₃₂ alkenyl groups with one or more C—C double bonds that include linear alkenyls, branched alkenyls with one or more branch points or linear or branched alkenyls which include one or more C₃-C₈ cycloalkane or cycloalkene groups, long-chain C₆-C₃₂ alkynyl groups with one or more C—C triple bonds that include linear alkynyls, branched alkynyls with one or more branch points or linear or branched alkynyls which include one or more C₃-C₈ cycloalkane groups or long-chain C₆-C₃₂ alkyl, alkenyl or alkynyl groups that are optionally substituted with aryl, halogen, alkoxy, mono- or di(C₁-C₆)amino, C₁-C₆-alkyl ester.

Suitable aralkyl groups of compounds of the invention include single and multiple ring compounds, including multiple ring compounds that contain separate and/or fused aryl groups. Typical aralkyl groups contain 1 to 3 separate or fused rings and from 6 to about 18 carbon ring atoms. Preferred aralkyl groups include benzyl and methylenenaphthyl (—CH₂-naphthyl), 1-phenethyl, 2-phenethyl, ω-phenyl-C₁₋₈alkyl, and other carbocyclic aralkyl groups, as discussed above.

“Alkoxy” means an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Preferred alkoxy groups are lower alkoxy groups having from 1 to about 6 carbon atoms.

The term “halogen” means fluorine, chlorine, bromine, iodine, or astatine.

As used herein, the term “metal ion” is intented to include any metal ion including all natural and synthetic isotopes thereof and further includes both radioactive and non-radioactive metal ions. The term radiolabelled typically refers to compounds or complexes comprising at least one radioactive isotope. In preferred embodiments of the invention, radiolabelled typically comprises complexes and compounds having at least one metal ion which is present as one or more isotopes of which at least isotope is radioactive.

### DETAILED DESCRIPTION OF THE INVENTION AND PREFERRED EMBODIMENTS THEREOF

The present invention provides new radiolabeled diagnostic and therapeutic agents which comprise a metal center. Preferred diagnostic agents comprise at least one radiometal, e.g., at least one radioactive isotope. Preferred therapeutic agents may comprise a radiolabelled or cold metal ions (e.g., isotopes of a metal which are not radioactive). Preferred radiometals include 99m-technetium and one or more radioactive isotopes of rhenium. Preferred agents of the present invention typically comprise an oxotechnetium core (Tc═O) or an oxorhenium core (Re═O) chelated by at least one ligand group Y linked to a tertiary amine pharmacophore as described in Formula I and Formula II supra. Preferred radiolabeled metal complexes of the invention comprise a neutral or cationic metal complex, e.g., a metal ion and the inner coordination sphere of ligands taken together are neutral or cationic. Preferably, the overall charge of the radiolabeled complex is either neutral or cationic.

The present invention provides small-molecule metal-complexes and methods of using such small molecule metal complexes as diagnostic and therapeutic probes for the non-invasive imaging and localization of proteins or receptors expressed (or over expressed ) in normal tissues and organs as well as identification of said receptors over expressed in certain diseases or tumors.

Particular proteins, receptors and neuroreceptors, such as serotonin receptors, including 5HT receptors, adrenoreceptors, including α₁ receptors, sigma receptors including σ₁ and σ₂ receptors, calcium channel receptors, emopamil binding proteins, adrenergic receptors, dopamine receptors, are implicated in various neurological disorders and are also over expressed in a variety of tumors or phathological conditions. The tetradentate N₂S₂ ^{99m}Tc-complexes and the corresponding rhenium complexes are linked via a linker to a tertiary amine, e.g., a substituted piperidine or a N-alkyl-N-((hetero)aryl)alkylamine, or the like, and possess affinity for 5HT_{1A}, sigma-1, sigma-2, Ca²⁺ channel receptors, EBP, or alpha-1 receptors expressed or over expressed on the cell surface or within the cell of neuronal cells or tumor cells.

Thus, the invention provides compounds according to the following Formula II:
Y—X—NR₁R₂
where Y is a chelating ligand capable of binding metal ion, X is a linking group containing a backbone chain having 1 to about 8 atoms, and R₁ and R₂ each are independently selected unsubstituted alkyl groups having from 1 to about 8 carbon atoms alkoxyalkyl groups having from about 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl, wherein at least one of R₁ or R₂ is a substituted alkyl group.

Preferred comounds of Formula II include those comounds wherein R₁ is an optionally substituted alkyl (preferably C₁₋₆alkyl), R₂ is an optionally substituted aryl or heteroaryl substituted alkyl (preferalby an (aryl)C₁₋₄alkyl or (heteroaryl)C₁₋₄alkyl), and X is an optionally substituted C₃₋₈alkylene (preferably a C₃₋₆alkylene).

Particularly preferred compounds of Formula II of the present invention include those compounds of Formula II-A: 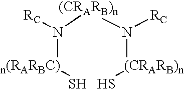
wherein:

R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;

R_{B} is hydrogen or a lower alkyl group having from 1 to about 6 carbon atoms for each occurrence of R_{B}; or

—(CR_{A}R_{B})— taken in combination is —(C═O)— such that there are zero or one —(C═O)— groups;

R_{C} is independently selected at each occurrence of R_{C} from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;

X is a linking group comprising a backbone chain having 1 to about 8 atoms, the backbone chain can optionally include ester, amide, ether or thioether linkages in the backbone chain; and

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl, wherein at least one of R₁ or R₂ is a substituted alkyl or alkyloxy group;

n is either 2 or 3 and is independently chosen at each occurrence of n; and

at least one occurrence of R_{A} or R_{C} in Formula I is chosen to be —XNR₁R₂,

where the metal complex resulting from the binding of the compound to the metal ion is either neutral or cationic.

Other preferred compounds of the present invention according to Formula I discussed supra include compounds according to Formulae I-A, I-B, I-C, and I-D:

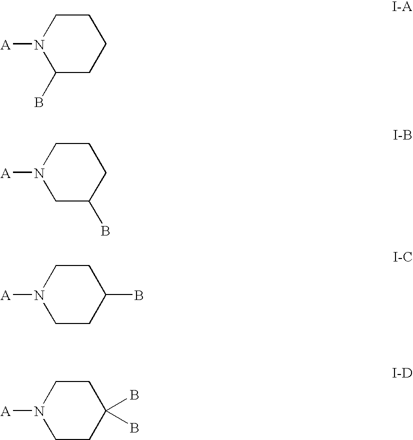

Radiolabeled complexes of the present invention can be isomerically pure or can comprise a mixture of isomers including mixtures of two or more isomers selected from enantiomers, diastereomers, complexation isomers, rotational isomers, geometric isomers, tautomers and like isomers. For example, isomeric complexes which result from the relative orientation of metal ligand group and a substitutents on the metal chelate group, Y, such as R_{A}, R_{C}, R, XNR₁R₂, X-(4-B—N-piperidinyl), or X—(N-A-piperidin-4-yl) are typically referred to as syn/anti isomers or alternatively as cis/trans isomers where the syn isomer has the oxo ligand and the ligand substituent oriented in generally the same direction and the anti isomer has the oxo ligand and the ligand substituent oriented in generally opposite directions.

Preferred metal ions for use in radiolabeled complexes of the invention are sources capable of emiting one or more discrete forms of radiation. Preferred radiation emissions include alpha, beta and gamma radiation emissions. Additionally preferred are metal ions that emit alpha, beta(+), beta(−) or gamma radiation with sufficient energy to be detected by standard radiography techniques or have sufficient alpha, beta or gamma energy for radiotherapeutic applications. Particularly preferred metal ions include one or more isotopes of metals selected from technetium, rhenium, ytttium, copper, gallium, indium, bismuth, platinum and rhodium. Technetium-99m and radioactive isotopes of rhenium are exemplary metal ion for use in the present invention. Metal ions suitable for use in radiolabeled complexes of the invention may include additional ligands coordinated to the metal atom. Preferred ligands include oxo, nitride, fluoride, chloride, bromide, iodide, carbonyl, isonitrile, nitrile, nitrosyl, alkoxide groups with 1 to about 6 carbon atoms, amine groups with 1 to about 12 carbon atoms, water, ether groups with 2 to about 8 carbon atoms, thioether groups with 2 to about 8 carbon atoms including thiophene, phosphines and phosphates with 1 to about 20 carbon atoms and other common ligands for technetium and rhenium chemistry. Particularly preferred technetium and rhenium metal ions additionally comprise an oxo ligand, e.g., a Tc═O or Re═O.

Additionally, preferred complexes of the invention have a chelating ligand moiety, Y, where the chelating ligand is able to bind to a metal ion through a plurality of donor atoms. Each donor atom is typically C, N, O, S, or P but other donor atoms are also acceptable for certain applications. Preferred donor atoms are N and S. The plurality of donor atoms can be present in a single compound or can be present in two or more compounds such that the two compounds bind to the metal to form the chelating ligand-metal complex. In certain embodiments, one compound will comprise three donor atoms and one or more additional compound will each independently comprise a single donor atom. Alternatively, two compounds, which can be the same or different, each of which can independently comprise two or more donor atoms can bind to a metal center to form a bis-igand metal complex.

Particularly preferred compounds and radiolabeled metal complexes comprise a tetradentate ligand system wherein the tetradentate ligand is contained in a single compound that includes four donor atoms. In additional preferred compounds and radiolabeled metal complexes, the tetradentate chelating ligand is a “3+1” ligand system wherein three donor atoms of the tetradentate chelating ligand moiety Y are contained in one compound and the fourth donor atom is present in another compound. Other chelating ligands, including bidentate, pentadentate, and ligands capable of chelating to two or more metal ions, are also contemplated for use in the compounds and metal complexes provided by the present invention.

Preferred linking groups, X, are lower alkyl groups having from 1 to about 8 atoms in the backbone such as, e.g., —(CH₂)ₙ—, ether groups having 3 to 8 atoms in the backbone such as, e.g., —(CH₂)ₙ—O—(CH₂)ₘ—, ester groups having 4 to 8 atoms in the backbone such as, e.g., —(CH₂)ₙ—CO—O—(CH₂)ₘ—, thioether groups having 3 to 8 atoms in the backbone such as, e.g., —(CH₂)ₙ—S—(CH₂)ₘ—, and amido groups having 4-8 atoms in the backbone such as, e.g., —(CH₂)ₙCO—NH—(CH₂)ₘ— where n and m are non-negative integers and the sum n+m is typically between about 2 and about 8. Particularly preferred linking groups X have between about 2 and about 5 atoms in the backbone.

Linking groups X may optionally have one or more substituents attached to the backbone chain including pendant aromatic groups. Preferred substituents include alkyl groups having from 1 to about 6 carbon atoms and from 0 to about 3 N, O or S atoms, hydroxyl, amino, carboxyl, alkoxy groups having from 1 to about 6 carbon atoms, aminoalkyl groups having from 1 to about 6 carbon atoms, dialkylaminoalkyl groups where each alkyl group has from about 1 to about 6 carbon atoms, halogen atoms including F, Cl, Br, and I, aromatic groups having about 5 to about 18 ring atoms which may include 0, 1, 2, or 3 N, O or S ring atoms.

Radiolabeled complexes of the invention include neutral or cationic metal centers where the metal center refers to the metal ion and the inner sphere of ligands directly bound to the metal ion. Preferred radiolabeled complexes of the invention contain a metal center that is neutral or cationic. Moreover, the radiolabeled complex comprising a metal ion and a compound of the formula Y—X—NR₁R₂ taken in its entirety is neutral or cationic.

Other preferred compounds provided by the invention according to Formula I and more preferably according to Formula I-C include the following compounds comprising a chelate, Y, according to Formula III: 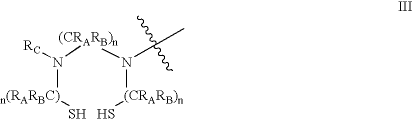
wherein:

R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;

R_{B} is hydrogen or lower alkyl having from about 1 to about 6 carbon atoms for each occurrence of R_{B}; or

—(CR_{A}R_{B})— taken in combination is —(C═O)— such that there are zero or one —(C═O)— groups;

R_{C} is selected from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;

X is a linking group comprising a backbone chain having 1 to about 8 atoms, the backbone chain can optionally include ester, amide, ether or thioether linkages in the backbone chain; and

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, optionally substituted heteroaryl;

n is either 2 or 3 and is independently chosen at each occurrence of n.

Preferred chelating groups according to Formula III include those chelates according to Formula III-A:

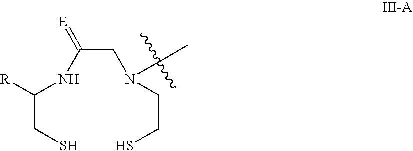

wherein

R is selected from hydrogen, COO(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms.

Particularly preferred X groups, e.g. linking groups between the amine pharmacophore and the metal chelate, in compounds according to any one of Formula I, I-A, I-B, I-C, I-D, II, or II-A include amide linkers of the formula, —(CH₂)ₘ—C(O)NH— (where m is between about 0 and about 5), and α,ω-alkylene groups wherein the alkylene group has between about 1 and about 10 carbon atoms and between 0 and about 3 oxygen or sulfur atoms in the alkylene chain.

The present invention further provides compounds according to Formula IV: 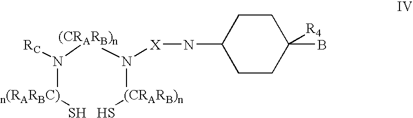
wherein:

B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;

R₄ is hydrogen, hydroxy, halogen, optionally substituted alkyl groups having from 1 to about 6 carbon atoms, optionally substituted alkoxy groups having from 1 to about 6 carbon atoms, or

R₄ and B taken in combination form an optionally substituted heterocyclic group having 5 or 12 ring atoms and one or two N, O, or S atoms and 1 or 2 fused rings;

R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;

R_{B} is hydrogen or lower alkyl having from 1 to about 4 carbon atoms for each occurrence of R_{B}; or

—(CR_{A}R_{B})— taken in combination is —(C═O)— such that there are zero or one —(C═O)— groups;

R_{C} is selected from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;

Y is a group capable of chelating to at least one metal ion;

X is a linking group comprising a backbone chain having 1 to about 8 atoms, the backbone chain can optionally include ester, amide, ether or thioether linkages in the backbone chain;

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and

n is either 2 or 3 and is independently chosen at each occurrence of n.

Particularly preferred compounds according to Formula IV provided by the present invention include those compounds according to Formula IV-A: 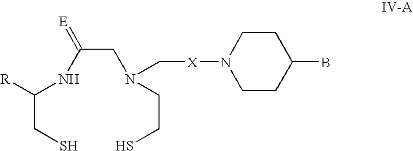
wherein:

B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;

Y is a group capable of chelating to at least one metal ion;

R is selected from hydrogen, C(O)O(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

The present invention further provides compounds according to Formula V: 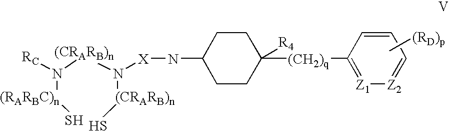
wherein:

R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, optionally substituted heteroalicyclic groups;

R₄ is hydrogen, hydroxy, halogen, optionally substituted alkyl groups having from 1 to about 6 carbon atoms, optionally substituted alkoxy groups having from 1 to about 6 carbon atoms, or

Z₁ and Z₂ are independently selected from CH, CR_{D}, and N;

p is selected from integers between about 0 and about 5;

q is selected from integers between about 0 and about 10;

R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;

R_{B} is hydrogen or lower alkyl having from about 1 to about 4 carbon atoms for each occurrence of R_{B}; or

—(CR_{A}R_{B})— taken in combination is —(C═O)— such that there are zero or one —(C═O)— groups;

R_{C} is selected from the group consisting of hydrogen, lower alkyl groups having 1 to about 8 carbon atoms, alkoxyalkyl groups having from 2 to 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;

Y is a group capable of chelating to at least one metal ion;

X is a linking group comprising a backbone chain having 1 to about 8 atoms, the backbone chain can optionally include ester, amide, ether or thioether linkages in the backbone chain;

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and

n is either 2 or 3 and is independently chosen at each occurrence of n.

Particularly preferred compounds according to Formula V provided by the present invention include those compounds according to Formula V-A: 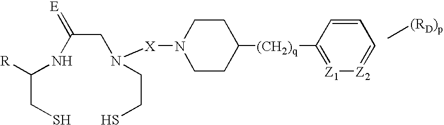
wherein:

R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, and optionally substituted heteroalicyclic groups;

Z₁ and Z₂ are independently selected from CH, CR_{D}, and N;

p is selected from integers between about 0 and about 5;

q is selected from integers between about 0 and about 10;

R is selected from hydrogen, C(O)O(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

The present invention further provides compounds according to Formula VI: 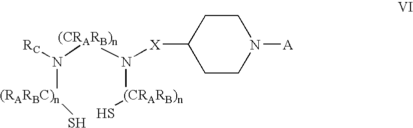
wherein:
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;

R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;

R_{B} is hydrogen or lower alkyl having from about 1 to about 4 carbon atoms for each occurrence of R_{B}; or

—(CR_{A}R_{B})— taken in combination is —(C═O)— such that there are zero or one —(C═O)— groups;

R_{C} is selected from the group consisting of hydrogen, lower alkyl groups having 1 to about 8 carbon atoms, alkoxyalkyl groups having from 2 to 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;

Y is a group capable of chelating to at least one metal ion;

X is a linking group comprising a backbone chain having 1 to about 8 atoms, the backbone chain can optionally include ester, amide, ether or thioether linkages in the backbone chain;

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and

n is either 2 or 3 and is independently chosen at each occurrence of n.

Particularly preferred compounds according to Formula VI provided by the present invention include those compounds according to Formula VI-A: 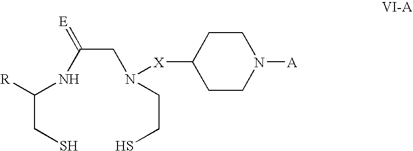
wherein:

A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;

R is selected from hydrogen, C(O)OR₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

In another embodiment, the present invention provides complexes wherein the metal complex is neutral or cationic that include a compound according to any one of Formula I, II, IV, V, VI or any subformula thereof and a metal ion. Additional preferred complexes comprise a metal ion and a compound of any of Formulas I, II, IV, V, VI, or any subformula thereof wherein the metal ion may comprise one or more radiolabeled isotopes or non-radiolabeled isotopes of the metal ion of the complex.

Preferred metal ions for use in radiolabeled complexes of the invention are sources of capable of emiting one or more discrete forms of radiation. Preferred radiation emissions include alpha, beta(+), beta(−), and gamma radiation emissions. Additionally preferred are metal ions that emit alpha, beta(+), beta(−), or gamma radiation with sufficient energy to be detected by standard radiography techniques or have sufficient alpha, beta(+), beta(−), or gamma energy for radiotherapeutic applications. Particularly preferred metal ions include one or more isotopes of metals selected from technetium, rhenium, ytttium, copper, gallium, indium, bismuth, platinum and rhodium. Technetium-99m and radioactive isotopes of rhenium, e.g., ¹⁸⁶Re and/or ¹⁸⁸Re, are exemplary radiolabeled metal ions for use in the radiolabled complexes and imaging methods using same provided by the present invention.

The present invention provides radiolabeled complexes comprising a compound according to Formula II or II-A and a metal ion. Particularly preferred complexes include complexes comprising a Tc or Re ion and a compound according to Formula II or II-A having a chelate Y according to Formula III-A and include those radiolabeled metal complexes according to Formula VII:

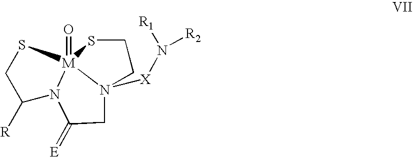

wherein

M is one or more isotopes of technetium or rhenium;

X is a linking group comprising a backbone chain having 1 to about 8 atoms, the backbone chain can optionally include ester, amide, ether or thioether linkages in the backbone chain; and

R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl, wherein at least one of R₁ or R₂ is a substituted alkyl or alkyloxy group;

R is selected from hydrogen, C(O)O(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl; and

E represents an oxo group or two hydrogen atoms.

Preferred complexes of Formula VII include those comounds wherein R₁ is an optionally substituted alkyl (preferably C₁₋₆alkyl), R₂ is an optionally substituted aryl or heteroaryl substituted alkyl (preferalby an (aryl)C₁₋₄alkyl or (heteroaryl)C₁₋₄alkyl), and X is an optionally substituted C₃₋₈alkylene (preferably a C₃₋₆alkylene).

The present invention additionally provides complexes comprising a compound according to Formula I and a metal ion. Preferred complexes include complexes comprising a compound according to Formula I-A, I-B or I-C having a chelate Y according to Formula III-A and a metal ion which may be radiolabeled or non-radiolabeled. Preferred radiolabeled complexes include those complexes comprising a compound according to Formula IV or IV-A and a metal ion, such as those metal complexes according to Formula VIII: 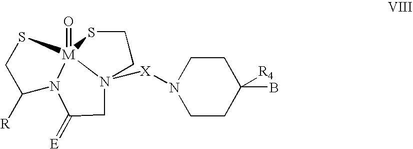
wherein

M is one or more isotopes of technetium or rhenium;

B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;

R₄ is hydrogen, hydroxy, halogen, optionally substituted alkyl groups having from 1 to about 6 carbon atoms, optionally substituted alkoxy groups having from 1 to about 6 carbon atoms, or

R₄ and B taken in combination form an optionally substituted heterocyclic group having 5 or 12 ring atoms and one or two N, O, or S atoms and 1 or 2 fused rings;

Y is a group capable of chelating to at least one metal ion;

R is selected from hydrogen, C(O)O(R₃), or C(O)N—H(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

Other preferred metal complexes comprise a compound according to Formula V or V-A and a metal ion such as those metal complexes according to Formula IX: 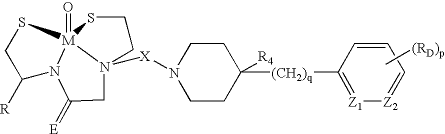
wherein:

M is one or more isotopes of technetium or rhenium;

R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, and optionally substituted heteroalicyclic groups;

R₄ is hydrogen, hydroxy, halogen, optionally substituted alkyl groups having from 1 to about 6 carbon atoms, optionally substituted alkoxy groups having from 1 to about 6 carbon atoms;

Z₁ and Z₂ are independently selected from CH, CR_{D}, and N;

p is selected from integers between about 0 and about 5;

q is selected from integers between about 0 and about 10;

R is selected from hydrogen, C(O)O(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

Other preferred metal complexes comprise a compound according to Formula VI or VI-A and a metal ion such as those metal complexes according to Formula X: 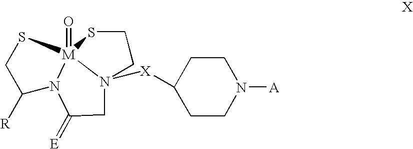
wherein:

M is one or more isotopes of technetium or rhenium;

A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;

R is selected from hydrogen, C(O)O(R₃), or C(O)NH(R₃);

R₃ represents hydrogen, optionally substituted alkyl, optionally substituted alkenyl, optionally substituted alkynyl, optionally substituted aryl, optionally substituted aralkyl, and optionally substituted cycloalkyl;

E represents an oxo group or two hydrogen atoms; and

X is a linking group comprising a backbone chain having 1 to about 8 atoms, the backbone chain can optionally include ester, amide, ether or thioether linkages in the backbone chain.

Particularly preferred radiolabeled complexes and non-radiolabelled complexes of the present invention include complexes having a Tc or Re ion and a compound selected from:

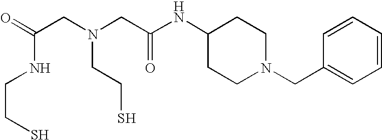

2-[[(1-Benzyl-piperidin-4-ylcarbamoyl)-methyl]-(2-mercapto-ethyl)-amino]-N-(2-mercapto-ethyl)-acetamide;

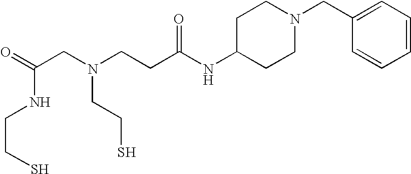

N-(1-Benzyl-piperidin-4-yl)-3-{(2-mercapto-ethyl)-[(2-mercapto-ethylcarbamoyl)-methyl]-amino}-propionamide;

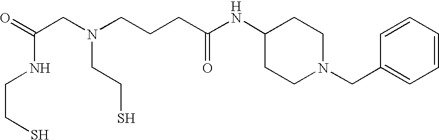

N-(1-Benzyl-piperidin-4-yl)-4-{(2-mercapto-ethyl)-[(2-mercapto-ethylcarbamoyl)-methyl]-amino}-butyramide;

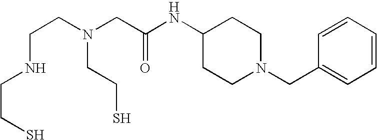

N-(1-Benzyl-piperidin-4-yl)-2-{(2-mercapto-ethyl)-[2-(2-mercapto-ethylamino)-ethyl]-amino}-acetamide;

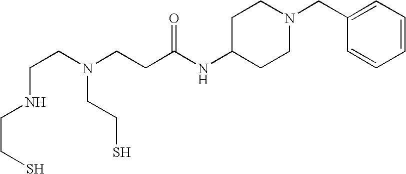

N-(1-Benzyl-piperidin-4-yl)-3-{(2-mercapto-ethyl)-[2-(2-mercapto-ethylamino)-ethyl]-amino}-propionamide;

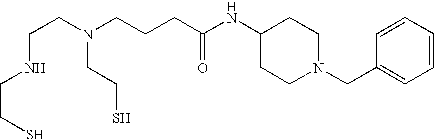

N-(1-Benzyl-piperidin-4-yl)-4-{(2-niercapto-ethyl)-[2-(2-mercapto-ethylamino)-ethyl]-amino}-butyramide;

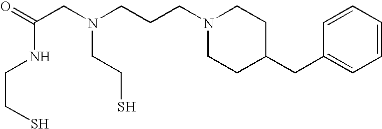

2-[[3-(4-Benzyl-piperidin-1-yl)-propyl]-(2-mercapto-ethyl)-amino]-N-(2-mercapto-ethyl)-acetamdide;

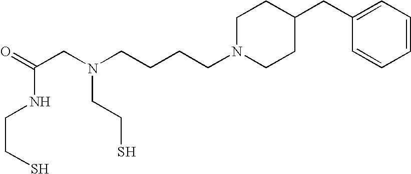

2-[[4-(4-Benzyl-piperidin-1-yl)-butyl]-(2-mercapto-ethyl)-amino]-N-(2-mercapto-ethyl)-acetamide;

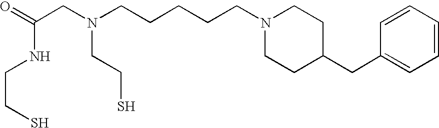

2-[[5-(4-Benzyl-piperidin-1-yl)-pentyl]-(2-mercapto-ethyl)-amino]-N-(2-mercapto-ethyl)-acetamide;

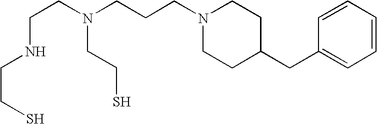

2-{2-[[3-(4-Benzyl-piperidin-1-yl)-propyl]-(2-mercapto-ethyl)-amino]-ethylamino}-ethanethiol;

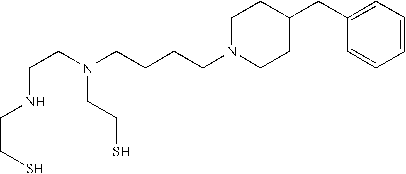

2-{2-[[4-(4-Benzyl-piperidin-1-yl)-butyl]-(2-mercapto-ethyl)-amino]-ethylamino}-ethanethiol;

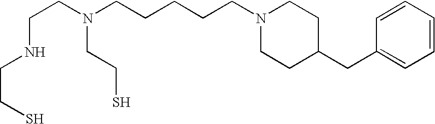

2-{2-[[5-(4-Benzyl-piperidin-1-yl)-pentyl]-(2-mercapto-ethyl)-amino]-ethylamino}-ethanethiol;

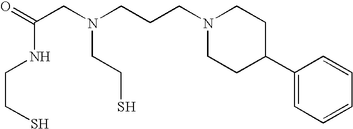

N-(2-Mercapto-ethyl)-2-{(2-mercapto-ethyl)-[3-(4-phenyl-piperidin-1-yl)-propyl]-amino}-acetamide;

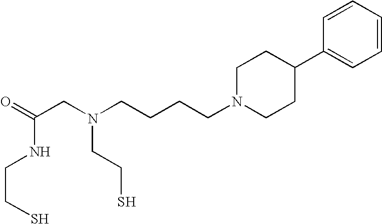

N-(2-Mercapto-ethyl)-2-{(2-mercapto-ethyl)-[4-(4-phenyl-piperidin-1-yl)-butyl]-amino}-acetamide;

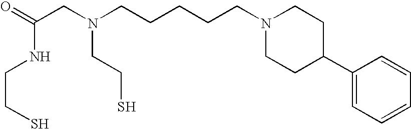

N-(2-Mercapto-ethyl)-2-{(2-mercapto-ethyl)-[5-(4-phenyl-piperidin-1-yl)-pentyl]-amino}-acetamide;

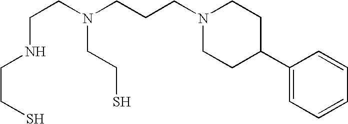

2-(2-{(2-Mercapto-ethyl)-[3-(4-phenyl-piperidin-1-yl)-propyl]-amino}-ethylamino)-ethanethiol;

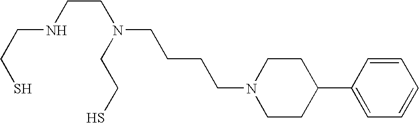

2-(2-{(2-Mercapto-ethyl)-[4-(4-phenyl-piperidin-1-yl)-butyl]-amino}-ethylamino)-ethanethiol;

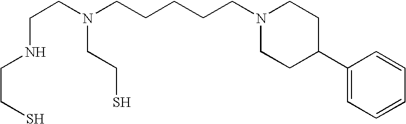

2-(2-{(2-Mercapto-ethyl)-[5-(4-phenyl-piperidin-1-yl)-pentyl]-amino}-ethylamino)-ethanethiol;

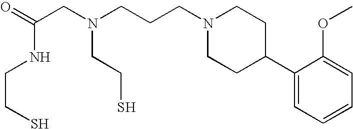

N-(2-Mercapto-ethyl)-2-((2-mercapto-ethyl)-{3-[4-(2-methoxy-phenyl)-piperidin-1-yl]-propyl}-amino)-acetamide;

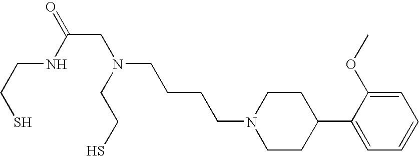

N-(2-Mercapto-ethyl)-2-((2-mercapto-ethyl)-{4-[4-(2-methoxy-phenyl)-piperidin-1-yl]-butyl}-amino)-acetamide;

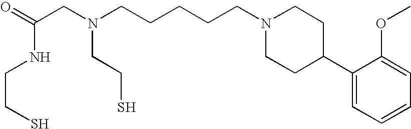

N-(2-Mercapto-ethyl)-2-((2-mercapto-ethyl)-{5-[4-(2-methoxy-phenyl)-piperidin-1-yl]-pentyl}-amino)-acetamide;

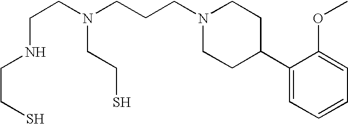

2-[2-((2-Mercapto-ethyl)-{3-[4-(2-methoxy-phenyl)-piperidin-1-yl]-propyl}-amino)-ethylamino]-ethanethiol;

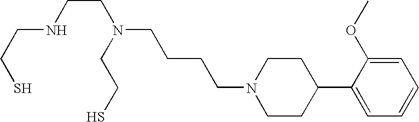

2-[2-((2-Mercapto-ethyl)-{4-[4-(2-methoxy-phenyl)-piperidin-1-yl]-butyl}-amino)-ethylamino]-ethanethiol;

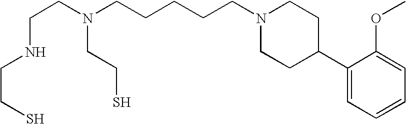

2-[2-((2-Mercapto-ethyl)-{5-[4-(2-methoxy-phenyl)-piperidin-1-yl]-pentyl}-amino)-ethylamino]-edmanethiol;

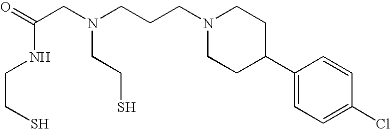

2-[{3-[4-(4-Chloro-phenyl)-piperidin-1-yl]-propyl}-(2-mercapto-ethyl)-amino]-N-(2-mercapto-ethyl)-acetamide;

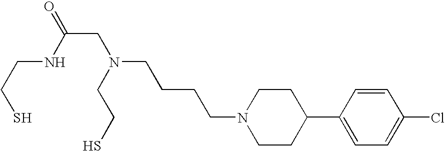

2-[{4-[4-(4-Chloro-phenyl)-piperidin-1-yl]-butyl}-(2-mercapto-ethyl)-amino]-N-(2-mercapto-ethyl)-acetamide;

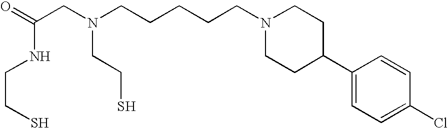

2-[{5-[4-(4-Chloro-phenyl)-piperidin-1-yl]-pentyl}-(2-mercapto-ethyl)-amino]-N-(2-mercapto-ethyl)-acetamide;

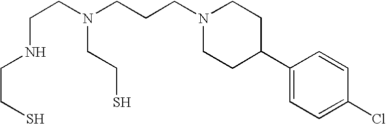

2-{2-[{3-[4-(4-Chloro-phenyl)-piperidin-1-yl]-propyl}-(2-mercapto-ethyl)-amino]-ethylamino}-ethanethiol;

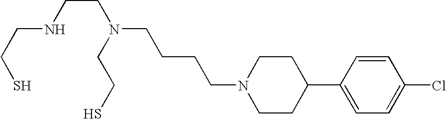

2-{2-[{4-[4-(4-Chloro-phenyl)-piperidin-1-yl]-butyl}-(2-mercapto-ethyl)-ethylamino}-ethanethiol;

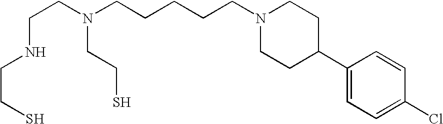

2-{2-[{5-[4-(4-Chloro-phenyl)-piperidin-1-yl]-pentyl}-(2-mercapto-ethyl)-amino]-ethylamino}-ethanethiol;

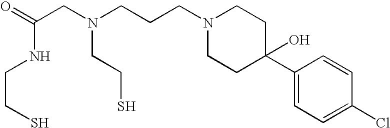

2-[{3-[4-(4-Chloro-phenyl)-4-hydroxy-piperidin-1-yl]-propyl}-(2-mercapto-ethyl)-amino]-N-(2-mercapto-ethyl)-acetamide

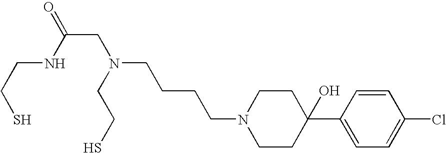

2-[{4-[4-(4-Chloro-phenyl)-4-hydroxy-piperidin-1-yl]-butyl}-(2-mercapto-ethyl)-amino]-N-(2-mercapto-ethyl)-acetamide;

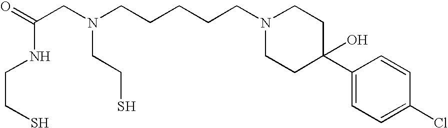

2-[{5-[4-(4-Chloro-phenyl)-4-hydroxy-piperidin-1-yl]-pentyl}-(2-mercapto-ethyl)-amino]-N-(2-mercapto-ethyl)-acetamide;

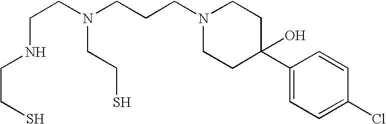

2-{2-[{3-[4-(4-Chloro-phenyl)-4-hydroxy-piperidin-1-yl]-propyl}-(2-mercapto-ethyl)-amino]-ethylamino}-ethanethiol;

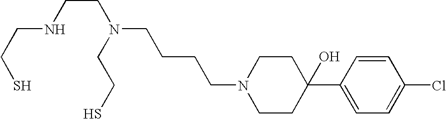

2-{2-[{4-[4-(4-Chloro-phenyl)-4-hydroxy-piperidin-1-yl]-butyl}-(2-mercapto-ethyl-amino]-ethylamino}-ethanethiol;

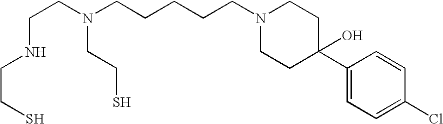

2-{2-[{5-[4-(4-Chloro-phenyl)-4-hydroxy-piperidin-1-yl]-pentyl}-(2-mercapto-ethyl)-amino]-ethylamino}-ethanethiol;

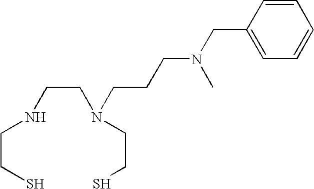

2-2-[[3-(Benzyl-methyl-amino)-propyl]-(2-mercapto-ethyl)-amino]-ethyloamino)-ethanethiol

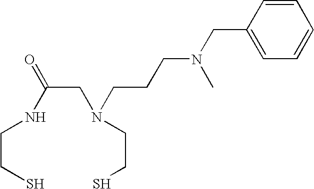

2-[[3-(Benzyl-methyl-amino)-propyl]-(2-mercapto-ethyl)-amino]-N-(2-mercapto-ethyl)-acetamide

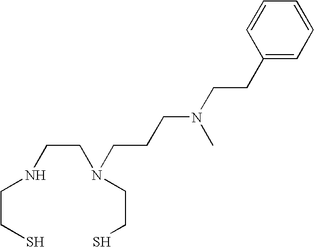

2-(2-{(2-Mercapto-ethyl)-[3-(methyl-phenethyl-amino)-propyl]-amino}-ethylamino)-ethanethiol

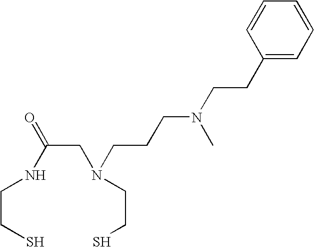

N-(2-Mercapto-ethyl)-2-{(2-mercapto-ethyl)-[3-(methyl-phenethyl-amino)-propyl]-amino}-acetamide

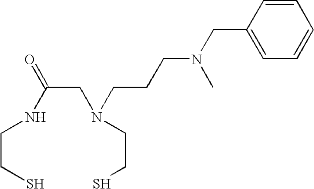

2-[[4-(Benzyl-methyl-amino)-butyl]-(2-mercapto-ethyl)-amino]-N-(2-mercapto-ethyl)-acetamide

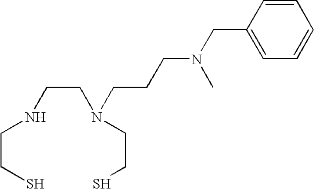

2-{2-[[4-(Benzyl-methyl-amino)-butyl]-(2-mercapto-ethyl)-amino]-ethylamino)-ethanethiol

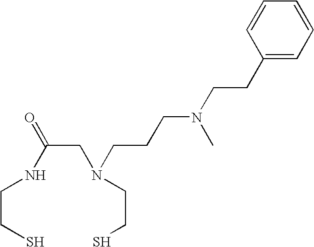

N-(2-Mercapto-ethyl)-2-{(2-mercapto-ethyl)-[4-(methyl-phenethyl-amino)-butyl]-amino}-acetamide

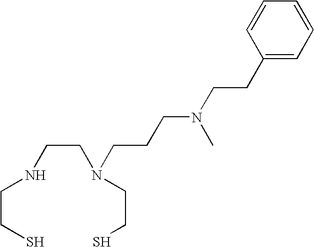

2-(2-Mercapto-ethyl)-[4-(methyl-phenethyl-amino)-butyl]-amino}-ethylamino)-ethanethio

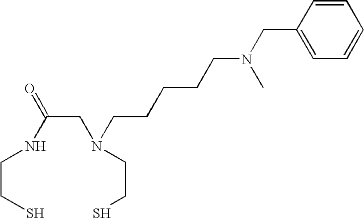

2-[[5-(Benzyl-methyl-amino)-pentyl]-(2-mercapto-ethyl)-amino]-N-(2-mercapto-ethyl)-acetamide

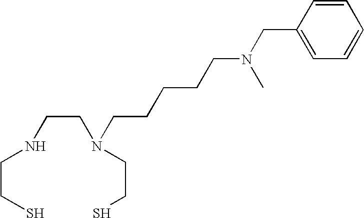

2-{2-[[5-(Benzyl-methyl-amino)-pentyl]-(2-mercapto-ethyl)-amino]-ethylamino}-ethanethiol

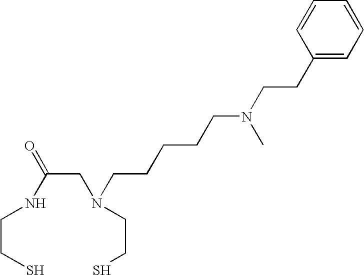

N-(2-Mercapto-ethyl)-2-{(2-mercapto-ethyl )-[5-(methyl-phenethyl-amino)-pentyl]-amino}-acetamide

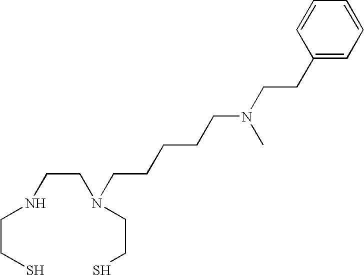

2-(2-{(2-Mercapto-ethyl)-[5-(methyl-phenethyl-amino)-pentyl]-amino}-ethylamino)-ethanethiol

Tumors suitable for imaging by the method of the present invention include neoplasms, carcinomas and other cancerous tumors. Preferred tumors for imaging include neoplasms of breast, prostate, lung, pancreas, liver, colon, lymphomas, gliomas, melanomas, and other neoplasms. Tumors, especially neoplasm and melanoma tumors, can be imaged in-vivo or in-vitro in any tissue. Preferably the tumor to be imaged is in a mammalian tissue, more preferably the tumor is in a human tissue. Preferred tissues and organs include skin, heart, brain, lung, spleen, colon, liver, kidney, muscle, lymph nodes, and other internal organs.

In theory any tissue, organ, tumor, growth of cells, bone, or other biomaterial may be imaged using the compounds, complexes or methods of the present invention provided that the radiolabeled metal complex used in the imaging methods is selectively taken up in the target tissue such that there is sufficient contrast between the tissue, organ, tumor, growth of cells, bone, or other biomaterial to be imaged and the background. Preferred tissue, organ, tumor, growth of cells, bone, or other biomaterial which are suitable for imaging using the compounds, metal complexes and imaging methods fo the present invention express or overexpress one or more receptors for which the compound or metal complex has an affinity.

Tissues suitable for imaging using the compounds and metal complexes or the methods of the invention are not particularly limited. Preferred tissues are capable of binding or taking up compounds of the present invention or are capable of retaining the compounds of the present invention to a greater extent than other tissues in the general vicinity of the tissue to be imaged. Thus, the emission of the radiolabeled complex retained in the tissue to be imaged has sufficient contrast against the other proximate tissues to allow for imaging of the tissue. Typically preferred tissues have one or more proteins and/or receptors to which the compounds of the present invention bind include one or more proteins, receptors or neuroreceptors, such as serotonin receptors, including 5HT receptors, adrenoreceptors, including α₁ receptors, sigma receptors including σ₁ and σ₂ receptors, calcium channel receptors, emopamil binding proteins, adrenergic receptors, dopamine receptorssubtypes and subclasses thereof and the like. More preferably, tissues comprise one or more receptors chosen from 5HT, including 5HT_{1A}, σ₁, σ₂, α₁, EBP, Ca²⁺ channel receptors, and the like.

The present invention provides preferred methods of imaging tumors in-vivo or in-vitro, the method comprising the steps of.

providing a radiolabeled complex comprising a compound of any one of Formula I, II, IV, V, VI or any subformula thereof and a metal ion or a radiolabeled metal complex of any one of Formula VIII, IX, X or any subformula thereof;

contacting the tumor(s) with the radiolabeled metal complex; and

making a radiographic image to image the tumor(s).

Particularly preferred tumor imaging methods provided by the present invention include those methods in which the radiolabeled complex comprises a metal ion and a compound of any one of claims Formula IV-A, V-A, or VI-A.

The present invention also provides preferred methods of imaging tissues or organs, particularly imaging of at least one tissue or organ expressing one or more receptors for which radiolabeled complexes have affinity, in-vivo or in-vitro, the method comprising the steps of:

providing a radiolabeled complex comprising a compound of any one of Formula I, II, IV, V, VI or any subformula thereof and a metal ion or a radiolabeled metal complex of any one of Formula VIII, IX, X or any subformula thereof;

contacting the tissue(s) or organ(s) expressing or overexpressing receptors with the radiolabeled metal complex; and

making a radiographic image to image the tissue(s).

In preferred embodiments, proteins and receptors are selected from serotonin receptors, α receptors, σ receptors, calcium channel receptors or emopamil binding proteins adrenergic receptors, adrenoceptors receptors, dopamine receptors, sigma receptors and any subclass of receptors or proteins thereof, more preferably the receptors are selected from 5HT_{1A}, σ₁, σ₂, α₁, EBP, Ca²⁺ channel receptors, and the like.

In other preferred embodiments of the invention, the tissue to be imaged is part of the central nervous system, particularly the brain or the spinal cord of a patient, or a tumor or organ which expresses one or more proteins or receptors to which one of the radiolabeled metal complexes of the invention have a binding affinity. Particularly preferred tissues include brain tissue which expresses one or more of proteins, receptors or neuroreceptors, particularly brain tissue expressing one or more of 5HT_{1A}, σ₁, σ₂, or α₁, EBP, Ca²⁺ channel receptors, and the like.

The present invention further provides methods for the treatment of cancer, the method comprising the steps of:

providing a cytotoxic metal complex comprising a metal ion and a compound of any one of of Formula I, II, IV, V, VI or any subformula thereof or a metal complex according to any one of Formula VIII, IX, X or any subformula thereof; and

contacting the tumor(s) with the cytotoxic metal complex.

Preferred methods of treatment of the invention contemplate the use of both cold metal complexes, e.g., non-radiolabeled metal complexes, and radiolabeled complexes for certain cancer therapies.

The present invention further provides methods of inhibiting a protein, receptor or neuroreceptor comprising the steps of

providing a metal complex comprising a metal ion and a compound of any one of claims 1-22 or a metal complex according to any one of claims 23-31; and

contacting the protein, receptor or neuroreceptor with the metal complex.

Preferred receptors or neuroreceptors which are suitable for inhibition by metal complexes of the invention include serotonin receptors, α receptors, σ receptors, calcium channel receptors or emopamil binding proteins adrenergic receptors, adrenoceptors receptors, dopamine receptors, and any subclass of receptors or proteins thereof, or more preferably include 5HT_{1A}, σ₁, σ₂, α₁, EBP, Ca²⁺ channel receptors, and the like.

The imaging and therapeutic methods of the invention generally comprise administration of an effective amount of one or more compounds of the invention to a subject including a mammal, such as a primate, especially a human, in need of such imaging or treatment. For imaging applications, typically a sufficient amount of a radiolabeled complex is administered to the tissue, organ, tumor, or the like to be imaged to provide for selective uptake of the radiolabeled complex into the tissue, organ or tumor to be imaged. Preferably the amount of radiolabeled complex taken up in the tissue, organ or tumor is sufficient to be imaged and/or quantified by standard radiographic techniques.

The treatment methods of the invention also will be useful for treatment of mammals other than humans, including for veterinary applications such as to treat horses and livestock e.g. cattle, sheep, cows, goats, swine and the like, and pets (companion animals) such as dogs and cats.

For diagnostic or research applications, a wide variety of mammals will be suitable subjects including rodents (e.g. mice, rats, hamsters), rabbits, primates and swine such as inbred pigs and the like. Additionally, for in vitro applications, such as in vitro diagnostic and research applications, body fluids (e.g., blood, plasma, serum, cellular interstitial fluid, saliva, feces and urine) and cell and tissue samples of the above subjects will be suitable for use.

Compounds of the invention may be administered singularly (i.e. sole therapeutic agent of a regime) or in combination with other agents for diagnostic ro therapeutic purposes which may or may not be radiolabeled to treat or prevent diseases and conditions such as undesired cell proliferation as disclosed herein. For combined diagnostic or therapeutic applications, additional agents are preferably chemotherapy agents or neurolyptic agents.

Pharmaceutical compositions of the invention include a compound of the invention packaged together with instructions (written) for therapeutic use of the compound, particularly to treat a subject suffering from or susceptible to tumors, e.g., cancers, such as melanoma, prostate cancer or the like. Pharmaceutical compositions of the invention may also be packaged together with instructions (written) for therapeutic use of the compound, particularly to image tissues or tumors within a subject to diagnose, identify or locate one or more tissues or tumors within the subject.

### EXAMPLES

General Experimental Details:

All chemicals and reagents, obtained from commercial sources (Aldrich Chemicals, Gibco Life Technologies), were of analytical grade and were used without further purification. ^{99m}Tc-pertechnetate was obtained via a generator (DuPont). ¹H NMR spectra were obtained on a Varian XL500 MHz instrument. Mass spectra were recorded on a MicroMass LCZ electrospray LC-MS instrument. HPLC purification was performed on a Waters Millennium Chromatography System equipped with a 996 UV-VIS diode-array detector attached in series to a gamma detector consisting of a shielded photomultiplier powered by a Canberra voltage amplifier and connected to a ratemeter. For the purification of all complexes, a reversed-phase C₈ column equipped with a C₁₈ guard was eluted with methanol (solvent A) and 0.005 M phosphate-buffered saline, pH 7.4, (Sigma) (solvent B) using a linear gradient from 15:85/A:B to 90:10/A:B at a 1.0 mL/min flow rate.

Synthesis of Ligands and Technetium and Rhenium Complexes.

AADT(Trt)2 chelate (1), N-3-chloropropyl-AADT (2), and AADT(Trt)₂-N-pentachlorophenylacetate (3) were synthesized as described earlier by us [Mahmood A, Kuchma M H, Freiberg E, Goldstone J, Davison A, Jones A G. Functionalized tetradentate N₂S₂ chelates and their technetium-99m and rhenium complexes: synthesis, spectroscopy and structural characterization. In: Nicolini M, Mazzi U, eds. Technetium, rhenium and other metals in chemistry and nuclear medicine 5. Padova: Servizi Grafici Editoriali, 1999:253-7.]

Technetium-99m-labeled complexes can be synthesized by transmetallation of technetium-99m from a prereduced ^{99m}Tc-glucoheptonate precursor (Scheme 2). Upon heating the reaction mixture at 70° C., ligand exchange of the AADT ligand bearing the pendant tertiary amines and the ^{99m}Tc(V)-glucoheptonate precursor yielded complexes Tc-(Complexes A-D and H-M) in nearly quantitative yields within 30 min. Typical mass amounts of the ^{99m}Tc-complexes preclude their physical characterization; however, since both technetium and rhenium form structurally identical AADT complexes, analogous non-radioactive rhenium complexes were synthesized (vide infra) and used as surrogates for HPLC comparisons. Identical HPLC retention times established the existence of the proposed technetium-99m species.

Using a method similar to that for ^{99m}Tc-complexes, the mono-oxorhenium(V) complexes (Examples 6-10) were obtained by reduction of perrhenate(VII) with stannous chloride in the presence of sodium glucoheptonate and the deprotected chelating ligand; heating the reaction mixture at 75° C. for 1 h afforded brownish-purple solids of the rhenium complexes. Upon chelation the N-substituent on the chelate may adopt a syn or anti configuration with respect to the asymmetric M=O core. The desheilding, anisotropic environment of the M=O core and the proximity of the N-substituent in the syn configuration to the asymmetric oxometal core results in a downfield shift of the proton resonances syn to the M=O core, thus permitting differentiation of the syn and anti diastereomers via NMR (Lever, S. Z.; Baidoo, K. E.; Mahmood, A. Structural Proof of Syn/Anti Isomerism in N-Alkylated Diaminedithiol (DADT) Complexes of Technetium. Inorg. Chim. Acta 1990, 176, 183-184; Francesconi, L. C.; Graczyk, G.; Wehrli, S.; Shaikh, S. N.; McClinton, D.; Liu, S.; Zubieta, J.; Kung, H. F. Synthesis and Characterization of Neutral M^{V}O (M=Tc, Re) Amine-Thiol Complexes Containing a Pendant Phenylpiperidine Group. Inorg. Chem., 1993, 32, 3114-3124; O'Neil, J. P.; Wilson, S. R.; Katzenellenbogen, J. A. Preparation and Structural Characterization of Monoamine-Monoamide Bis(Thiol) Oxo Complexes of Technetium(V) and Rhenium(V). Inorg. Chem., 1994, 33, 319-323; and Pelecanou, M.; Chryssou, K.; Stassinopoulou, C. I. Trends in NMR Chemical Shifts and Ligand Mobility of TcO(V) and ReO(V) Complexes with Aminothiols. J. Inorg. Biochem., 2000, 79,347-351).

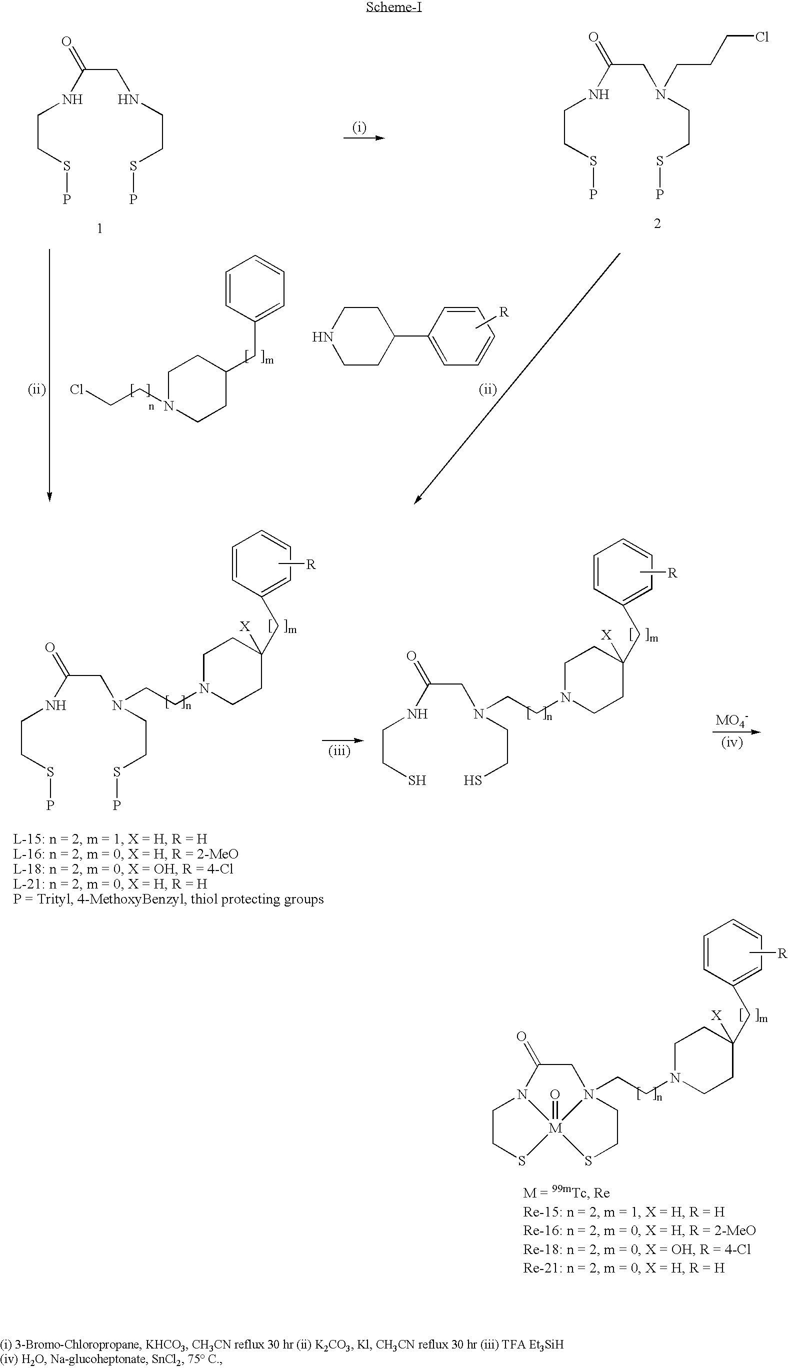

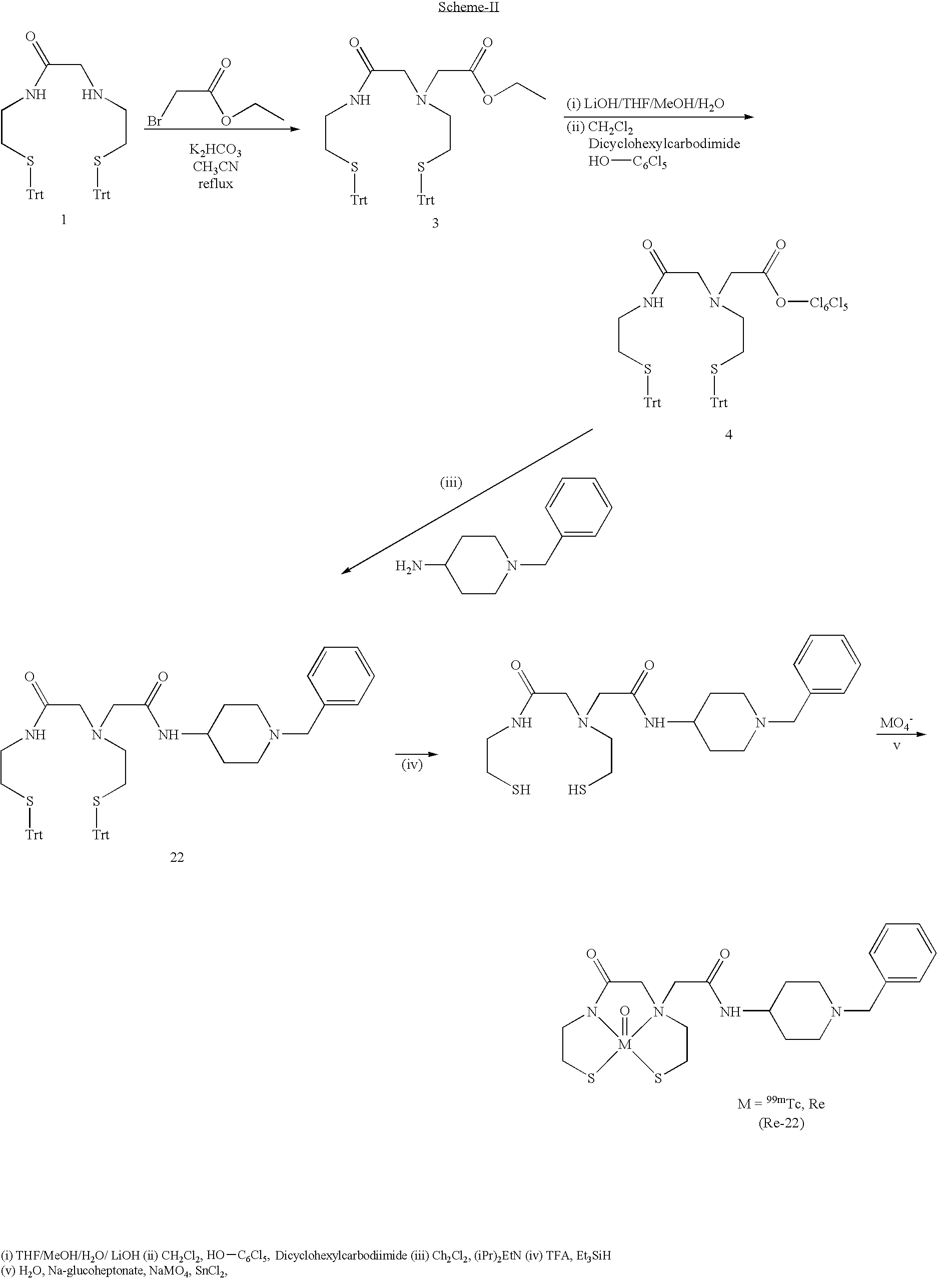

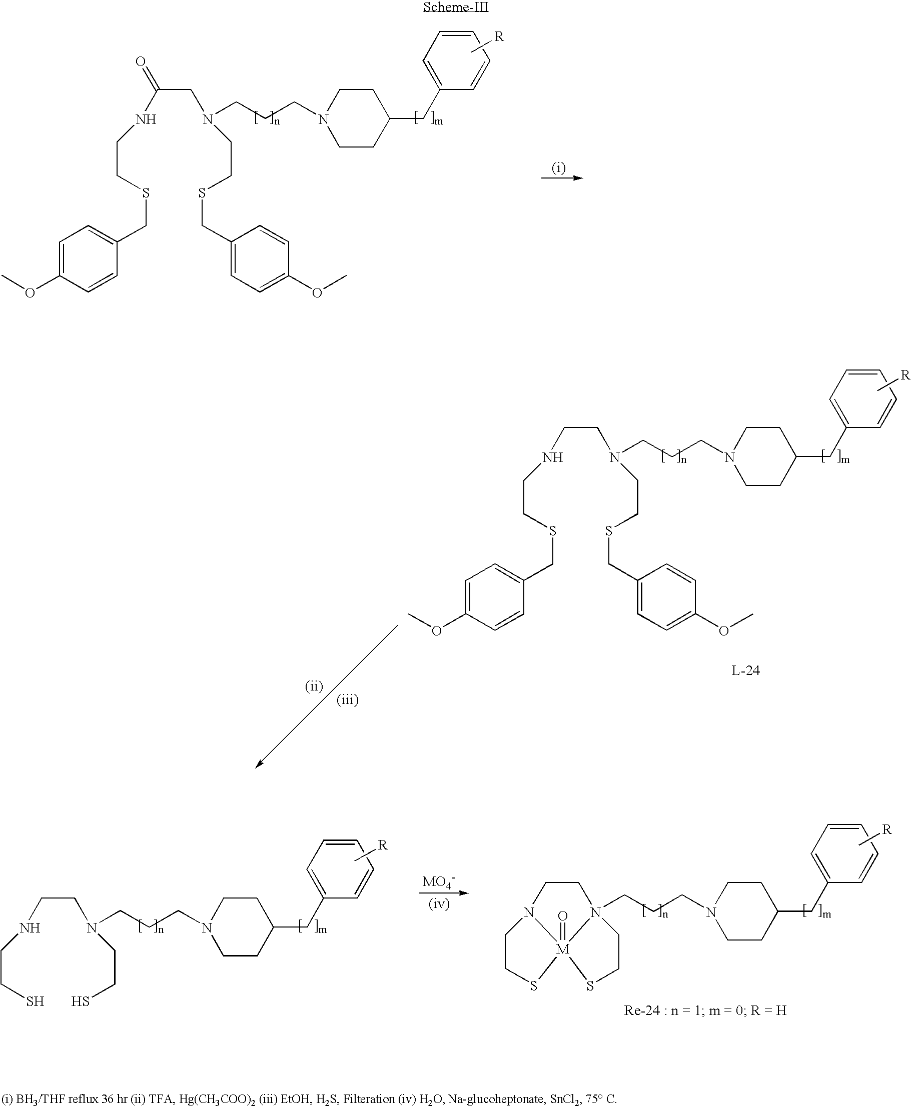

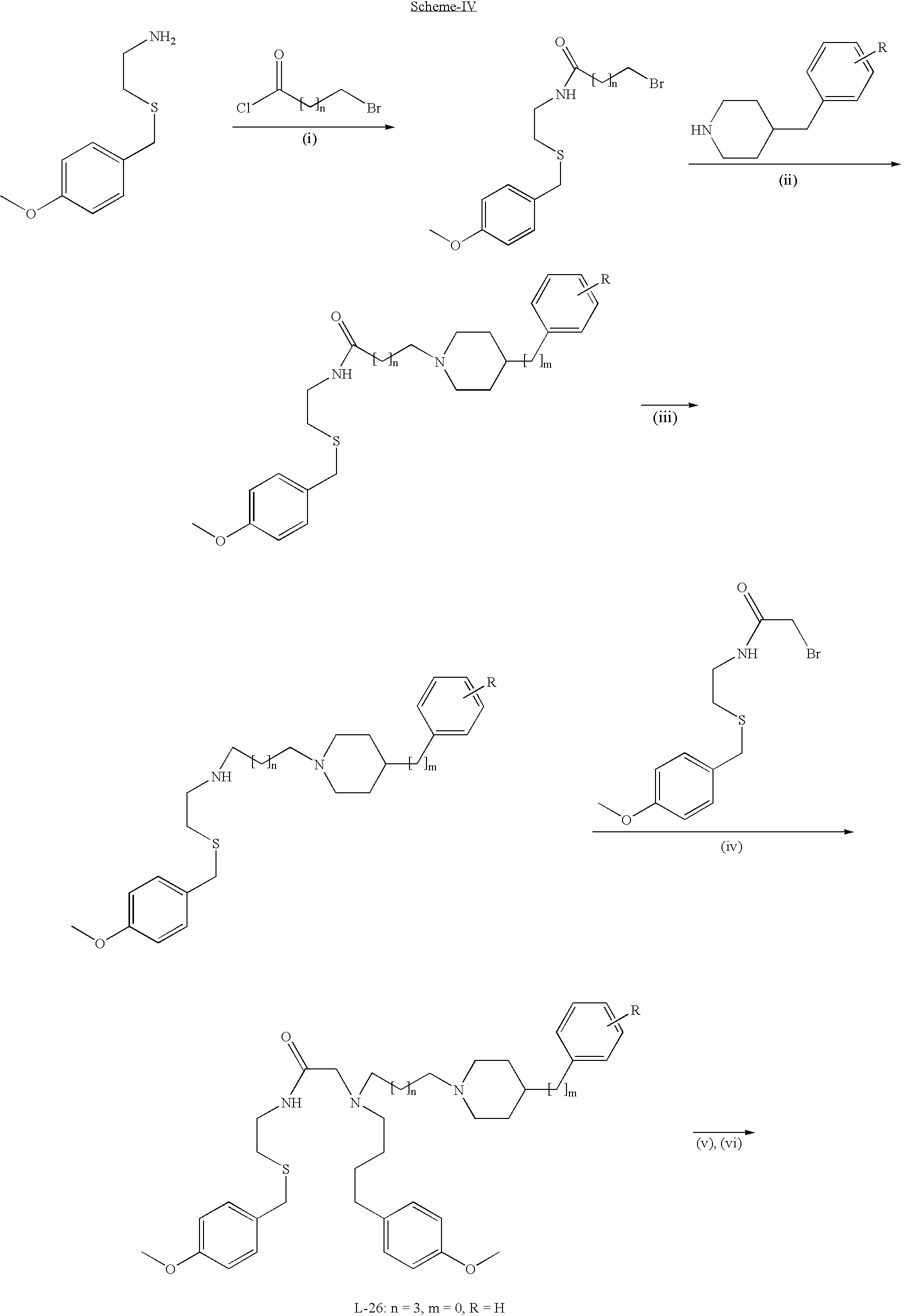

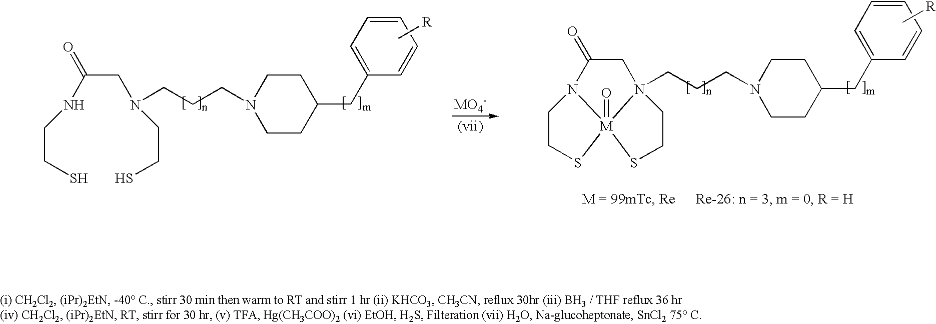

### Example 1

### 4-Benzyl, N—(CH₂)₃-(AADT(Trt)₂)-piperidine (Ligand 15)

AADT(Trt)2 chelate (1) (0.25 g, 0.37 mmol) was dissolved with N-(3 chloropropyl),4-benzylpiperidine (0.2 g, 0.79 mmol) in dry acetonitrile. K₂CO₃ (0.55g, 3.95 mmol) and KI (0.66 g, 3.97 mmol) was added to this solution and the reaction mixture was refluxed for 30 hr under argon. The solvent was evaporated from the reaction mixture to dryness and redissolved in CH₂Cl₂ followed by filteration to remove the solids. The filterate was evaporated and the crude pale yellow oil was chromatographed on silica with first with CH₂Cl₂ followed by 4% methanol in CH2Cl2 to yield a pale yellow oil (0.188 g, 0.21 mmol, 56.8%)

¹H NMR (CDCl₃): 7.5-7.34 (m, 12H, Ar), 7.33-7.05 (m, 23H, Ar), 3.09-2.8 (q, 2H, —CH₂), 2.8-2.7 (m, 4H, —CH₂), 2.6-2.45 (d, 2H, —CH₂), 2.45-2.15 (m, 10H, —CH₂), 1.95-1.7 (t, 2H, —CH₂), 1.65-1.4 (m, 5H, —CH₂), 1.35-1.15 (m, 2H, —CH₂).

Mol. Wt: 894.28, C₅₉H₆₃N₃OS₂, C, 79.2%; H, 7.1%; N 4.7%.

Exact Mass: 893.44, ESI mass Spec (M+H)⁺=894.43

### Example 2

### 4(2-MeOphenyl)-N—(CH₂)₃-(AADT(Trt)₂)-piperidine (Ligand 16)

N-3-chloropropyl-AADT (2) (0.3 g, 0.397 mmol) was dissolved in acetonitrile along with 4-Methoxyphenyl piperidine (0.114 g, 0.595 mmol). K₂CO₃ (0.275 g, 1.98 mmol) and KI (0.33 g, 1.98 mmol) was added to this solution and the reaction mixture was refluxed for 30 hr under argon. The solvent was evaporated from the reaction mixture to dryness and redissolved in CH₂Cl₂ followed by filteration to remove the solids. The filterate was evaporated and the crude pale yellow oil was chromatographed on silica with first with CH₂Cl₂ followed by 4-5% methanol in CH₂Cl₂ to yield a pale yellow oil (0.29 g, 0.318 mmol, 80.24%)

¹H NMR (CDCl₃): 7.54-7.32 (m, 12H Ar), 7.32-7.1 (m, 20H, Ar), 7.0-6.91 (d, 1H, Ar), 6.91-6.8 (d, 1H, Ar), 3.82 (s, 3H, OCH₃), 3.12-2.88 (m, 5H, —CH₂), 2.85 (s, 2H, CO—CH₂), 2.5-2.18 (10H, —CH₂), 2.1-1.9 (m, 2H, —CH₂), 1.86-1.7 (m, 2H, —CH₂), 1.64-1.5 (m, 2H—CH₂).

Mol. Wt: 910.28, C₅₉H₆₃N₃O₂S₂, C, 77.85%; H, 6.98%; N, 4.62%.

Exact Mass: 909.44, ESI mass Spec (M+H)⁺=910.28

### Example 3

### 4-Hydroxy,4-(4Chlorophenyl)-N—(CH₂)₃-(AADT(Trt)₂)-piperidine (Ligand 18)

AADT(Trt)2 chelate (1) (0.35 g, 0.515 mmol) was dissolved with N-(3 chloropropyl),4-hydroxy, 4-phenylpiperidine (0.223 g, 0.77 mmol) in dry acetonitrile. K₂CO₃ (0.53 g, 3.85 mmol) and KI (0.255 g, 1.54 mmol) was added to this solution and the reaction mixture was refluxed for 30 hr under argon. The solvent was evaporated from the reaction mixture to dryness and redissolved in CH₂Cl₂ followed by filteration to remove the solids. The filterate was evaporated and the crude pale yellow oil was chromatographed on silica with first with CH₂Cl₂ followed by 5% methanol in CH₂Cl₂ to yield a pale yellow oil (0.279 g, 0.3 mmol, 58%)

¹H NMR (CDCl₃): 7.46-7.31 (m, 13H, Ar), 7.31-7.08 (m, 21H, Ar), 3.15-2.95 (m, 3H, —CH₂), 2.9-2.7 (m, 3H, —CH₂), 2.7-2.45 (m, 3H, —CH₂), 2.45-2.0 (m, 12H, —CH₂), 1.65-1.5 (m, 3H, —CH₂).

Mol. Wt: 930.70, C₅₈H₆₀CIN₃O₂S₂, C, 74.85%; H, 6.5%; N, 4.5%.

Exact Mass: 929.38, ESI mass Spec (M+H)⁺=929.99

### Example 4

### (4-phenyl)-N—(CH₂)₃-(AADT(Trt)₂)-piperidine (Ligand 21)

N-3-chloropropyl-AADT (2) (0.35 g, 0.463 mmol) was dissolved in acetonitrile along with 4-phenyl piperidine (0.11 g, 0.682 mmol). K₂CO₃ (0.32 g, 2.3 mmol) and KI (0.38 g, 2.31 mmol) was added to this solution and the reaction mixture was refluxed for 30 hr under argon. The solvent was evaporated from the reaction mixture to dryness and redissolved in CH₂Cl₂ followed by filteration to remove the solids. The filterate was evaporated and the crude pale yellow oil was chromatographed on silica with first with CH₂Cl₂ followed by 4-5% methanol in CH₂Cl₂ to yield a pale yellow oil (0.327 g, 0.372 mmol, 80.3%)

¹H NMR (CDCl₃): 7.58-7.35 (m, 13H, Ar), 7.34-7.12 (m, 22H, Ar), 3.12-2.94 (m, 4H, —CH₂), 2.88 (s, 2H), 2.6-2.2 (m, 11H, —CH₂), 2.14-1.88 (m, 2 H, —CH₂), 1.88-1.73 (m, 4 H, —CH₂), 1.72-1.5 (m, 2 H, —CH₂).

Mol. Wt: 880.26, C₅₈H₆₁N₃OS₂, C, 79.14%; H, 6.98%; N, 4.77%.

Exact Mass: 879.43, ESI mass Spec (M+H)⁺=880.37

### Example 5

### N-benzyl, 4-amidocarboxy-(CH2)-AADT(Trt)₂-piperidine (Ligand 22)

AADT(Trt)₂-N-pentachlorophenylacetate (3) (0.21 g, 0.213 mmol) was dissolved in dry CH₂Cl₂ and N-benzyl,4-aminopiperidine (0.049 g, 0.25 mmol) was added to this solution along with diisopropylethylamine (0.033 g, 0.255 mmoles). The reaction was allowed to stir at room temperture for 5 hr after which the crude was reduced in volume and chromatographed on silica with 6% methanol in CH₂Cl₂ to yield a offwhite solid (0.188 g, 0.206 mmol, 97%)

¹H NMR (CDCl₃): 7.6-7.26 (m, 17H, Ar), 7.26-7.08 (m, 16H, Ar), 7.06-6.88 (d, 1H, Ar), 6.7-6.58 (m, 1H, Ar), 3.8-3.6 (m, 2 H, —CH₂), 3.45 (s, 2 H, —CH₂), 3.14-2.86 (m, 6 H, —CH₂), 2.8-2.6 (m, 2 H, —CH₂), 2.6-2.47 (m, 2 H, —CH₂), 2.46-2.35 (t, 2 H, —CH₂), 2.32-2.22 (m, 2 H, —CH₂), 2.14-1.92 (m, 2 H, —CH₂), 1.9-1.68 (m, 2 H, —CH₂), 1.54-1.3 (m, 2 H, —CH₂).

Mol. Wt: 909.25, C₅₈H₆₀N₄O₂S₂, C, 76.6%; H, 6.65%; N, 6.16%.

Exact Mass: 908.42, ESI mass Spec (M+H)⁺=909.10

### Example 6

### Re Complex of Ligand 15

Ligand 15 (0.16 g, 0.179 mmol) was dissolved in 20 mL Trifluroacetic acid and the yellow color was titrated with Et₃SiH till the solution became colorless. The deprotected ligand solution was evaporated to dryness to remove residual acid and re-dissolved in 30-40 mL degassed distilled water. To this solution was added sodium glucoheptonate (0.122 g, 0.492 mmol) and sodium perrhenate (0.067 g, 0.245 mmol) followed by adjusting the pH to 5 with NaOH. Solid SnCl₂ (0.092 g, 0.485 mmol) was then added and the solution stirred at 70° C. for 1 hr. The pH was readjusted to 5-6 and heated for an additional 2 hr at 70° C. After allowing the solution to come to room temperature the pale purple aqueous solution was extracted with CH₂Cl₂ ( 15 mL×3) to yield the crude rhenium complex. Chromatography on silica with 3-4% methanol in CH₂Cl₂ yielded the pale purple rhenium complex Re-15 (0.0855 g, 0.14 mmol, 78%).

¹H NMR (CDCl₃): 7.38-7.18 (m, 3H, Ar), 7.18-7.08 (m, 2H, Ar), 4.663 (d, 1H), 4.565 (m, 1H), 4.18-4.05 (m, 2H), 3.96 (dd, 1H), 3.594 (ddd, 1H), 3.37 (ddd, 1H), 3.28-3.06 (m, 3H), 2.94-2.79 (m, 3H), 2.53 (d, 2H), 2.44-2.28 (m, 2H), 2.08-1.83 (m, 4H), 1.74-1.4 (m, 4H), 1.4-1.18 (m, 2H).

Mol. Wt.: 608.84, C₂₁H₃₂N₃O₂ReS₂, C, 41.43%; H, 5.3%; N, 6.9%.

Exact Mass: 609.15, ESI mass Spec (M+H)⁺=610.01

### Example 7

### Re Complex of Ligand 16

The complex was synthesized using a procedure similar to that described for Re-15 using ligand-16 (0.15 g, 0.164 mmol), sodium-glucoheptonate (0.082 g, 0.33 mmol), NaReO4 (0.045 g, 0.164 mmol) and SnCl₂ (0.062 g, 0.328 mmol). The pale purple complex was isolated by silica chromatography by eluting with 4% Methanol in CH₂Cl₂ (0.0645 g, 0.1 mmol, 62%)

¹H NMR (CDCl₃): 7.24-7.12 (m, 2H Ar), 7.0-6.8 (m, 2H, Ar), 4.69 (d, 1H), 4.56 (m, 1H), 4.1 (d, 1H), 4.12-3.92 (m, 2H), 3.81 (s, 3H, OCH₃), 3.639 (ddd, 1H), 3.41 (ddd, 1H), 3.32-3.12 (m, 3H), 3.12-2.92 (m, 3H), 2.89 (ddd 1H), 2.457 (dd, 1H), 2.32-2.08 (m, 3H), 2.08-1.905 (m, 2H), 1.9-1.7 (m, 3H), 1.61 (ddd 1H).

Mol. Wt.: 624.84, C₂₁H₃₂N₃O₃ReS₂, C, 40.37%; H, 5.16%; N, 6.72%.

Exact Mass: 625.14, ESI mass Spec (M+H)⁺=625.86

### Example 8

### Re Complex of Ligand 18

The complex was synthesized using a procedure similar to that described for Re-15 using ligand-18 (0.11 g, 0.118 mmol), sodium-glucoheptonate (0.059 g, 0.237 mmol), NaReO4 (0.0323 g, 0.118 mmol) and SnCl₂ (0.046 g, 0.242 mmol). The pale purple complex was isolated by silica chromatography by eluting with 4% Methanol in CH₂Cl₂ (0.0418 g, 0.065 mmol, 55%).

¹H NMR (CDCl₃): 7.455 (m, 1H, Ar), 7.42 (m, 1H, Ar), 7.345 (m, 1H, Ar), 7.303 (m, 1H, Ar), 4.683 (d, 1H), 4.577 (m, 1H), 4.2-3.9 (m, 3H), 3.657 (ddd, 1H), 3.38 (ddd, 1H), 3.3-3.08 (m, 3H), 2.94-2.68 (m, 3H), 2.64-2.38 (m, 4H), 2.24-1.88 (m, 4H), 1.84-1.66 (m, 2H), 1.66-1.5 (m, 1H)

Mol. Wt.: 645.25, C₂₀H₂₉CIN₃O₃ReS₂, C, 37.23%; H, 4.53%; N, 6.51%.

Exact Mass: 645.09, ESI mass Spec (M+H)⁺=645.64.

### Example 9

### Re Complex of Ligand 21

The complex was synthesized using a procedure similar to that described for Re-15 using ligand-21 (0.175 g, 0.199 mmol), sodium-glucoheptonate (0.098 g, 0.395 mmol), NaReO4 (0.081 g, 0.296 mmol) and SnCl₂ (0.15 g, 0.79 mmol). The pale purple complex was isolated by silica chromatography by eluting with 4% Methanol in CH₂Cl₂ (0.07 g, 0.117 mmol, 59.2%)

¹H NMR (CDCl₃): 7.4-7.14 (m, 5H, Ar), 4.696 (d, 1H), 4.56 (m, 1H), 4.118 (d, 1H), 4.1-3.9 (m, 2H), 3.64 (ddd, 1H), 3.405 (ddd, 1H), 3.32-3.1 (m, 3H), 3.1-2.94 (m, 2H), 2.6 (dd, 1H), 2.64-2.3 (m, 3H), 2.24-1.92 (m, 5H), 1.92-1.71 (m, 3H), 1.617 (ddd, 1H).

Mol. Wt.: 594.81, C₂₀H₃₀N₃O₂ReS₂, C, 40.38%; H, 5.08%; N, 7.06%.

Exact Mass: 595.13, ESI mass Spec (M+H)⁺=595.76.

### Example 10

### Re Complex of Ligand 22

The complex was synthesized using a procedure similar to that described for Re-15 using ligand-22 (0.10 g, 0.11 mmol), sodium-glucoheptonate (0.054 g, 0.22 mmol), NaReO4 (0.045 g, 0.165 mmol) and SnCl₂ (0.082 g, 0.43 mmol). The pale purple complex was isolated by silica chromatography by eluting with 5% Methanol in CH₂Cl₂ (0.051 g, 0.081 mmol, 74%)

¹H NMR (CDCl₃): 7.36-7.32 (m, 5H, Ar), 6.238 (d, 1H, NH), 4.955 (d, 1H), 4.692 (d, 1H), 4.7-4.52 (m, 2H), 4.26-4.08 (m, 2H), 3.92 (dd, 1H), 3.803 (m, 1H), 3.537 (s, 2H), 3,428 (ddd, 1H), 3.35-3.04 (m, 2H), 3.05-2.74 (m, 3H), 2.3-2.2 (m, 3H), 2.0-1.8 (m, 2H), 1.7-1.4 (m, 3H).

Mol. Wt.: 623.81, C₂₀H₂₉N₄O₃ReS₂, C, 38.5%; H, 4.69%; N, 8.98%.

Exact Mass: 624.12, ESI mass Spec (M+H)⁺=624.93.

### Example 11

### General Procedure for Deprotection of Trityl Protected Thiol Groups

6.0 mg of the bis-trityl-protected AADT-ligand was dissolved in 3 ml of trifluoro acetic acid and stirred at room temperature for 5 min. 1-2 drops of triethylsilyl hydride were added until the former yellowish reaction mixture became colorless.

The solvent was evaporated completely and the residue placed under high vacuum overnight.

The synthesis of the technetium and rhenium labeled complexes is outlined in Scheme 1.

### Example 12

### Technetium-99m Labeling

Technetium-99m labeling was performed using 1.0 mg of the thiol-deprotected ligands (Compound A-D, F or H-M) dissolved in 0.5 ml phosphate buffer (0.005 M, pH=7.5), which were exchange-labeled with the required activity of ^{99m}Tc-glucoheptonate by heating the reaction at 60-75° C. for 45 min. HPLC evaluation of the technetium-99m-labeled complexes showed 80-95% radiochemical yield.

Co-injection of the characterized rhenium complexes with the analogous technetium-99m complexes showed co-elution of the radioactive species with the corresponding UV active rhenium complex.

### Example 13

### General Procedure for Rhenium Complexation

The bistrityl-protected ligand (Compound A-D, or G-M) (100 mg, 0.1 mmol) was dissolved in 0.25 ml anisol and 10 ml trifluoroacetic acid. The resulting yellow solution was stirred for 5 min and then titrated with triethylsilyl hydride until colorless. The solution was evaporated and placed on high vacuum till completely dry residue remained. The residue was redissolved in 5 ml 20% MeOH in water previously argon-saturated. To this solution was added an aqueous solution of NaReO₄ (30 mg, 0.1 mmol) and Na-glucoheptonate (55 mg, 0.22 mmol) and, while stirring, solid SnCl₂ (21 mg, 0.11 mmol). The solution began to turn a brownish purple color. The pH of the reaction mixture was adjusted to 7 and the reaction was heated at 75° C. for 1 hr. The solution was then cooled to room temperature and the pH was adjusted to 8, followed by extraction with CH₂Cl₂. The CH₂Cl₂ extract was concentrated and chromatographed on silica gel, eluting with 4% MeOH in CH₂Cl₂ to yield the desired product as a pale purple solid.

### Example 14

### 5HT_{1A} Receptor Assays

The in vitro 5HT_{1A} binding affinities of rhenium coordinated complexes were determined in a competition assay using rat hippocampus and high-affinity 5HT_{1A}-ligand [³H]-8-OH-DPAT (135 Ci/mmol, NEN Life Science Inc., Cambridge, Mass.). See, Brain Res. 1995, 673, 217-225.

Male Sprague-Dawley rats (weighing 150-170 g) were sacrificed using anesthesia agent isoflurane. The brains were rapidly removed, and hippocampus, frontal cortex, hypothalamus, and striatum were hand-dissected on ice and stored at −70° C. Tissue was thawed at room temperature and homogenized using a Brinkmann Polytron tissue disrupter in 50 volumes (wt/vol) of ice-cold 50 mM Tris-HCl buffer (pH 7.4). The suspension was centrifuged twice at 27,000 g for 20 min at 4° C. The membrane pellets were resuspended in 50 volumes of (wt/vol) Tris-HCl buffer and incubated at 37° C. for 20 min in a water bath, before a final centrifugation step (27,000 g; 20 min; 4° C.). The final tissue pellets were stored at −70° C. until assayed.

Twelve concentrations of the nonradioactive rhenium complexes ranging from 1×10⁻¹¹ to 1×10⁻⁴ and protein samples (0.15 mg of membrane protein) were incubated with 1.5 nM [³H]-8-OH-DPAT in a total volume of 0.25 mL of Tris-HCl (50 mM, pH 7.4, 10 mM MgSO₄). Incubations were carried out for 60 min at 25° C. All assays were terminated by dilution with 5 mL of ice-cold Tris-HCl (10 mM), pH 7.4, and solution were filtered through glass-fiber filters (Whatman GF/F; presoaked in 0.5% polyethyleneimine for 30 min at 25° C.). Filters were then washed three times with 5 mL of ice-cold Tris-HCl (50 mM, pH 7.4), and counted in Hionic-Fluor cocktail (Packard, Groningen, the Netherlands). The corresponding IC₅₀ values were determined with Origin 6.0 software (OriginLab, Northampton, Mass.) and were used for the calculation of the apparent Kᵢ values with the Cheng-Prusoff equation. See, Biochem. Pharmacol. 1973, 22, 3099-3108.

### Example 15

### Alpha-1, α₁ Receptor Assays

The in vitro α₁ receptor binding affinities of rhenium coordinated complexes were determined in a competition assay using rat frontal cortex and high-affinity α₁ ligand [³H]-Prazosin (80 Ci/mmol, NEN Life Science Inc., Cambridge, Mass.). See, Eur. J. Nucl. Med. 2002, 29, 82-87.

The frontal cortex of rat brain was prepared as described above and store at −70° C. until used in the binding assays. Ten concentrations of the nonradioactive rhenium complexes ranging from 1×10⁻¹⁰ to 1×10⁻³ and protein samples (0.15 mg of membrane protein) were incubated with 1.5 nM [³H]-Prazosin in a total volume of 0.25 mL of Tris-HCl (50 mM, pH 7.4, 10 mM MgSO₄). Incubations were carried out for 60 min at 25° C. All assays were terminated by dilution with 5 mL of ice-cold Tris-HCl (10 mM), pH 7.4, and solution were filtered through glass-fiber filters (Whatman GF/F; presoaked in 0.5% polyethyleneimine for 30 min at 25° C.). Filters were then washed three times with 5 mL of ice-cold Tris-HCl (50 mM, pH 7.4), and counted in Hionic-Fluor cocktail (Packard, Groningen, the Netherlands). The corresponding IC₅₀ values were determined with Origin 6.0 software (OriginLab, Northampton, Mass.) and were used for the calculation of the apparent Kᵢ values with the Cheng-Prusoff equation. See, Biocheni. Pharmacol. 1973, 22, 3099-3108.

### Example 16

### Sigma-1, σ₁ Receptor Assays

The in vitro σ₁ receptor binding affinities of rhenium coordinated complexes were determined in a competition assay using rat frontal cortex and high-affinity σ₁ ligand [3H]-(+)-pentazocine (28 Ci/mmol, NEN Life Science Inc., Cambridge, Mass.). See, Mol. Neuropharmacol. 1993, 3, 117-126.

The membranes were prepared from guinea pig brain (minus cerebellum) as described above and stored at −70 ° C. Twelve concentrations of the nonradioactive rhenium complexes ranging from 1×10⁻¹¹ to 1×10⁻³ and protein samples (0.15 mg of membrane protein) were incubated with 5 mM [3H]-(+)-pentazocine in a total volume of 0.25 mL of Tris-HCl (50 mM, pH 8.0). Incubations were carried out for 120 min at 25° C. All assays were terminated by dilution with 5 mL of ice-cold Tris-HCl (10 mM), pH 8.0, and solution were filtered through glass-fiber filters (Whatman GF/F; presoaked in 0.5% polyethyleneimine for 30 min at 25° C.). Filters were then washed three times with 5 mL of ice-cold Tris-HCl (50 mM, pH 8.0), and counted in Hionic-Fluor cocktail (Packard, Groningen, the Netherlands). The corresponding IC₅₀ values were determined with Origin 6.0 software (OriginLab, Northampton, Mass.) and were used for the calculation of the apparent Kᵢ values with the Cheng-Prusoff equation. See, Biochem. Pharmacol. 1973, 22, 3099-3108.

### Example 17

### σ₂ Receptor Binding Assays

Rat liver membranes were prepared from male Sprague-Dawley rat livers as previously described (Eur. J. Pharmacol.—Mol. Pharmacol. Sect. 1994, 268, 9-18). The in vitro σ₂ receptor binding affinities of rhenium coordinated complexes were determined in a competition assay using rat livers and [³H]-DTG (31 Ci/mmol, NEN Life Science Inc., Cambridge, Mass.) as radioligand in the presence of 10 μM 1-pyrrolidinylethyl 3,4-dichlorophenylacetate oxalate (ACT915 oxalate) to mask σ₁ receptors (Bioorg. & Med. Chem. Lett. 2000, 10, 17-18). Competition assays were performed with twelve concentrations of the nonradioactive rhenium complexes ranging from 1×10⁻¹⁰ to 1×10⁻³ and protein samples (0.15 mg of membrane protein) in a total volume of 0.25 mL of Tris-HCl (50 mM, pH 8.0) for 120 min at 25° C. All other manipulations and data analysis were performed as described vide supra for the σ₁-receptor assays.

### Example 18

### In-Vivo Tumor Uptake

To study the tumor uptake of radiolabeled metal complexes, in vivo, biodistribution experiments at 1 h after their administration were carried out in C57B16 male mice with palpable B16 melanoma nodules and male nude mice bearing DU145 human prostate carcinoma in the hind limb.

The biodistribution data including tumor/nontumor (T/NT) ratios for selected organs are summarized in Table 1 and 2 as percentage injected dose per gram (% ID/g).

### Example 19

### Determination of Lipophilicity and pKₐ Values

The lipophilicity and pKₐ values of all complexes were determined using HPLC methods described previously (Stylli, C.; Theobald, A. E. Determination of Ionization Constants of Radiopharmaceuticals in Mixed Solvents by HPLC. Appl. Radiat. Isot., 1987, 38, 701-708; Johannsen, B.; Scheunemann, M.; Spies, H.; Brust, P.; Wober, J.; Syhre, R.; Pietzsch, H.-J. Technetium(V) and Rhenium(V) Complexes for 5-HT_{2A} Serotonin Receptor Binding: Structure-Affinity Considerations. Nucl. Med. Biol., 1996, 23, 429-438; and Johannsen, B.; Berger, R.; Brust, P.; Pietzsch, H.-J.; Scheunemann, M.; Seifert, S.; Spies, H.; Syhre, R. Structural Modification of Receptor-Binding Technetium-99m Complexes in Order to Improve Brain Uptake. Eur. J. Nucl. Med. 1997, 24, 316-319). Log P, log D_{(pH 7.4)} and pKₐ values were determined on a Perkin-Elmer HPLC system 1020 using a reversed phase PRP-1 column (250×4.1 mm; 10 μm; Hamilton) run under isocratic conditions with a flow rate of 1.5 mL/min at room temperature. The mobile phase was acetonitrile:phosphate buffer (0.01 M), 3:1, v/v, with the aqueous buffer adjusted to the desired pH between 3 and 11. The capacity factor (k′) was calculated for each determination (Braumann, T.; Grimme, L. H. Determination of Hydrophobic Parameters for Pyridazinone Herbicides by Liquid-Liquid Partition and Reversed-Phase High-Performance Liquid Chromatography. J. Chromatogr. 1981, 206, 7-15; El Tayer, N.; van der Waterbeemd, H.; Testa, B. Lipophilicity Measurements of Protonated Basic Compounds by Reversed-Phase High-Performance Liquid Chromatography. II. Procedure for the Determination of a Lipophilic Index Measured by Reversed-Phase High Performance Liquid Chromatography. J. Chromatogr. 1985, 320, 305-312; and Minick, D. J.; Frenz, J. H.; Patrick, M. A.; Brent, D. A. A Comprehensive Method for Determining Hydrophobicity Constants by Reversed-Phase High-Performance Liquid Chromatography. J. Med. Chem., 1988, 31, 1923-1933) and the partition coefficient at a given pH (D or logD) were calculated from the equation: log D=a log k′+b where the parameters a and b are predetermined using standard amines. The fitted points of inflection from the sigmoidal D_{HPLC}/pH profiles permit calculation of the pK_{HPLC}(Stylli, C.; Theobald, A. E. Determination of Ionization Constants of Radiopharmaceuticals in Mixed Solvents by HPLC. Appl. Radiat. Isot., 1987, 38, 701-708). The aqueous ionization constants pKₐ were calculated from the pK_{HPLC} values after correction with a predetermined correction factor obtained using standard amine compounds. Log P values of the neutral complexes were estimated from the respective upper plateau of the sigmoidal log D/pH curve in the alkaline range.

### Example 20

### Emopamil Binding Protein (EBP) Binding Assay

Guinea-pig liver membranes-homogenates are prepared following the procedure described by Christina Zech et al (European Journal of Pharmacology-Molecular Pharmacology section, 208: 119-130 (1991) and Fabian F. Moebius et al ( Molecular Pharmacology 43: 139-148, 1993). The binding assays can be preformed following the procedure described in the above two references. Briefly, in a total volume of 1.0 mL buffer (containing 0.1 w/v digitonin, 10 mM tris-HCl, 0.1 mM PMSF, pH 7.4) are suspended 0.03-0.04 mg of guinea-pig liver microsomal membranes, 0.5 nM (±)-[³H]emopamil, the reference drug or Re-complex (in concentration's ranging from 10⁻³ M to 10⁻¹² M). After incubation at room temperature for 1-2 hr, the binding is terminated by the addition of 3.0 mL of ice-cold buffer (10% w/v PEG 6000, 10 mM Tris-HCl, 10 mM MgCl₂) pH 7.4 and vacuum filtration through GF/F filters that are presoaked in PEI (0.5% for 20 min). The filters are then washed with an additional 3.0 mL buffer and placed in vials. Following addition of 10.0 mL scintillation liquid, (Hionic-Fluor cocktail, Packard, Groningen, the Netherlands) the amount of [³H]emopamil bound to the membranes is determined and can be plotted against the concentration of the Re-complex or drug reference. The corresponding IC₅₀ values can be determined with Origin 6.0 software (OriginLab, Northampton, Mass.) and are used for the calculation of the apparent Kᵢ values using the Cheng-Prusoff equation (Cheng, Y.; Prusoff, W. H. Biochem. Pharmacol. 22, 3099-3108, 1973).

### Example 21

### Ca⁺² Channel Binding Assay

The Ca⁺² channel affnnity for the reference drug or Re-complexes can be determined by the procedure described by Francesco Berardi et al (Bioorganic & Medicinal Chemistry, 9: 1325-1335, 2001). Briefly, rat brain membrane-preparation can be obtained by the procedure described by Ian J. Reynolds et al (J. Pharmacology and Experimental Therapeutics, 237(3): 731-738, 1986). The 0.05 to 0.1 mg of brain-membranes so obtained are suspended in a total volume of 1.0 mL of 50 mM Hepes buffer pH 7.4, along with 0.2 nM [³H]-desmethoxyverapamil and the reference drug or Re-complex (in concentration's ranging from 10⁻³ M to 10⁻¹² M). After incubation at room temperature for 1 hr, the assay is terminated by rapid filteration on GF/F filters that are presoaked in PEI (0.5%) and washed twice with 1.0 mL of ice-cold buffer. The filters are placed in scintillation vials and following addition of 10.0 mL scintillation liquid, (Hionic-Fluor cocktail, Packard, Groningen, the Netherlands) the amount of [³H]-desmethoxyverapamil bound to the membranes is determined and can be plotted against the concentration of the Re-complex or drug reference. The corresponding IC₅₀ values can be determined with Origin 6.0 software (OriginLab, Northampton, Mass.) and are used for the calculation of the apparent Kᵢ values using the Cheng-Prusoff equation (Cheng, Y.; Prusoff, W. H. Biochem. Pharmacol. 22, 3099-3108, 1973).

### Example 22

### Preparation of Ligand 24: N,N′-Bis-[2-(4-methoxy-benzylsulfanyl)-ethyl]-N-[3-(4-phenyl-piperidin-1-yl)-propyl]-ethane-1,2-diamine

Ligand 24 was prepared by the synthetic procedure depicted in Scheme III.

¹H NMR (CDCl₃): 7.34-7.15 (9H,m, Ar), 6.9-6.75 (4H,d, Ar), 3.77 (6H, br-s, OCH₃), 3.65(4H, br-s, CH₂-Ph), 3.02(3H, m, CH₂), 2.88-2.7 (3H, m, CH₂), 2.67-2.25 (16H,m, CH₂), 2.2-1.9 (5H,m, CH₂). C₃₆H₅₁N₃O₂S₂: Exact Mass: 621.34; MassSpec (ES⁺): 622.2 (M+H⁺)

### Example 23

### Preparation of Ligand 25: 2-[[3-(Methyl-phenethyl-amino)-propyl]-(2-tritylsulfanyl-ethyl)-amino]-N-(2-tritylsulfanyl-ethyl)-acetamide

Ligand 25 was prepared by a synthetic procedure depicted in Scheme 1 in which N-methyl N-(2-phenylethyl)amine is used as a nucleophile in place of tie 4-substituted piperidine in step (ii).

¹H NMR (CDCl₃): 7.51(1H,t,), 7.44-7.31 (12H, m, Ar), 7.28-7.11 (23H,m, Ar), 3.01 (2H, quart, CH₂—Ar), 2.82 (2H,s, CO—CH₂), 2.699(2H,m, CH₂), 2.53 (2H,m, CH₂), 2.45-2.3 (6H, m, CH₂), 2.295-2.22 (4H,m, CH₂), 2.19 (3H,m, CH₃), 1.488 (2H,m, CH₂). C₅₆H₅₉N₃OS₂:

Exact Mass: 853.41; MassSpec. (ESI⁺): 854.8 (M+H)⁺

### Example 24

### Preparation of Ligand 26: N-[2-(4-Methoxy-benzylsulfanyl)-ethyl]-2-{[2-(4-methoxy-benzylsulfanyl)-ethyl]-[5-(4-phenyl-piperidin-1-yl)-pentyl]-amino}-acetamide was prepared by the synthetic procedure depicted in Scheme IV

¹H NMR (CDCl₃): 7.77(1H,m), 7.4-7.1 (8H,m, Ar), 6.9-6.75 (4H,d,Ar), 3.58-3.55(6H,br-s, OCH₃), 3.66 (2H,s, CH₂-Ph), 3.63 ((2H, s, CH₂-Ph), 3.42 (2H, q, CH₂), 3.05 (1H, m, CH), 3.022 (2H, s, CO—CH₂), 2.7-2.3 (10H, m CH₂), 2.2-1.95 3H, m, CH₂), 1.95-1.7 (3H, m CH₂), 1.6-1.19 (7H, m, CH₂). C₃₈H₅₃N₃O₃S₂: Exact Mass=663.35; MassSpec (ESI^{+ ):} 664.13 (M+H)⁺

### Example 25

### Re Complex of Ligand 24 (Re-24)

The complex was synthesized using a procedure similar to that described for Re-15 using ligand-24 obtained as described in Scheme III.

¹H NMR (CDCl₃): 7.4-7.15 (5H, m, Ar), 4.3-4.0 (3H, m, CH₂), 4.0-3.7 (2H, m, CH₂), 3.7-3.5 (1H, m, CH), 3.5-3.15 (4H, m, CH₂), 3.15-2.85 (4H, m, CH₂), 2.75 (1H, dd, CH), 2.65-2.3 (3H, m, CH, CH₂), 2.25-1.95 (4H, m, CH₂), 1.94-1.78 (4H, m, CH₂), 1.77-1.66 (1H, m, CH). C₂₀H₃₂N₃OReS₂: Exact Mass=581.15; MassSpec (ESI⁺): 582.32 (M+H)⁺

### Example 26

### Re Complex of Ligand 25 (Re-25)

The complex was synthesized using a procedure similar to that described for Re-15 using ligand-25

¹H NMR (CDCl₃): 7.4-7.1 (5H, m, Ar), 4.539 (1H, m, CH), 4.366 (1H, d, CO—CH,), 4.072 (1H, m, CH), 3.924 (1H, d, CO—CH_{b}), 3.786 (1H, m, CH), 3.491 (1H, m CH), 3.385-3.0 (4H, m, CH₂), 2.9-2.7 (3H, m, CH₂), 2.68-2.54 (2H, m, CH₂), 2.322 (3H, s, CH₃), 1.827 (2H,m, CH₂), 1.55 (1H, ddd, CH).

C₁₈H₂₈N₃O₂ReS₂: Exact Mass=569.12; MassSpec (ESI⁻): 570.06 (M+H)⁺.

### Example 27

### Re Complex of Ligand 26 (Re-26)

The complex was synthesized using a procedure similar to that described for Re-15 usingligand-26 which was obtained as described in Scheme IV.

¹H NMR (CDCl₃): 7.4-7.15 (5H, m, Ar), 4.655 (1H, d, CO—CHₐ), 4.59 (1H, m, CH), 4.2-3.84 (3H, d+m, CO—CH_{b}, CH₂), 3.7-3.14 (5H, m, CH₂), 3.14-2.95 (2H, bt-d, CH₂), 2.96-2.74 (1H, dd, CH), 2.62-2.3 (3H, m, CH₂), 2.2-1.95 (2H, ddd, CH₂), 1.95-1.72 (8H, m, CH₂), 1.7-1.5 (3H, m, CH, CH₂).

C₂₂H₃₄N₃O₂ReS₂: Exact Mass 623.16; MassSpec (ESI⁺): 624.01 (M+H)⁺.

TABLE 1
In-vitro Receptor Affinity, pKₐ and Lipophilicity of Rhenium complexes for various receptors

| | σ₁ | σ₂ | α₁ | 5HT_{1A} | pKa | k' | k' |
|---|---|---|---|---|---|---|---|
| Compound | kᵢ(μM) | kᵢ(μM) | kᵢ(μM) | kᵢ(nM) | (Corrected) | (Max) | (pH = 7.4) |
| Re-15 | 0.547 | 0.136 ± 0.022 | 0.322 ± 0.061 | 589 ± 70 | 8.34 | 160 | 60 |
| Re-16 | 4.83 | 0.336 ± 0.07 | 0.048 ± 0.009 | 4.5 ± 0.4 | 8.09 | 117 | 57 |
| Re-18 | 2.96 | 0.528 ± 0.111 | 0.980 ± 0.18 | N.D. | 7.26 | 37.5 | 29.7 |
| Re-21 | 0.553 | 0.0846 ± 0.0079 | 0.039 ± 0.004 | 55.7 ± 8.3 | | | |
| Re-22 | 5.511 | 4.707 ± 0.986 | N.D. | N.D. | | | |
| Re-24 | 0.0203 ± 0.0015 | 0.0680 ± 0.0032 | 0.4236 ± 0.0553 | 423 ± 70 | | | |
| Re-25 | 3.652 ± 0.542 | 0.351 ± 0.0155 | 2.476 ± 0.903 | 329 ± 27 | | | |
| Re-26 | 0.0347 ± 0.0018 | 0.0483 ± 0.0067 | 0.125 ± 0.0261 | 707.6 ± 98.5 | | | |

N.D. = not determined

TABLE 2
One Hour Biodistribution of ^{99m}Tc-15 in subcutaneously
transplanted tumors in mice

| | Human Prostate CA | | Melanoma | |
|---|---|---|---|---|
| | (DU145) | | (B16/F0) | |
| Organs | %ID/g | S.D. | %ID/g | S.D. |
| Blood | 0.31 | 0.01 | 0.44 | 0.08 |
| Heart | 1.82 | 0.22 | 1.87 | 0.42 |
| Liver | 12.06 | 2.22 | 10.23 | 1.25 |
| Lung | 9.96 | 0.56 | 15.60 | 2.69 |
| Muscle | 1.06 | 0.14 | 0.90 | 0.06 |
| Kidney | 7.92 | 1.69 | 7.66 | 2.28 |
| Spleen | 7.56 | 1.28 | 5.95 | 1.07 |
| Brain | 0.51 | 0.06 | 0.48 | 0.1 |
| Intestine | 20.94 | 15.56 | 14.38 | 8.87 |
| Stomach | 5.26 | 2.16 | 6.81 | 2.33 |
| Skin | 1.76 | 0.21 | 1.21 | 0.45 |
| Tumor | 2.46 | 0.28 | 3.18 | 0.56 |
| Tumor/Blood | 7.93 | 0.91 | 7.47 | 2.48 |
| Tumor/Muscle | 2.33 | 0.23 | 3.57 | 0.76 |
| Tumor/Liver | 0.21 | 0.03 | 0.21 | 0.07 |
| Tumor/Lung | 0.25 | 0.02 | 0.31 | 0.07 |

The present invention has been described in detail. However, it will be appreciated that those skilled in the art may make modifications and improvements within the scope of the invention. For example, the pharmacore group may be linked to a carbon atom of the chelating ligand instead of to a nitrogen atom.

1. A compound capable of binding a metal ion, the compound according to the formula: 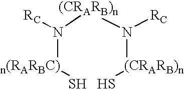
wherein:
R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;
R_{B} is hydrogen or a lower alkyl group having from 1 to about 6 carbon atoms for each occurrence of R_{B}; or
—(CR_{A}R_{B})— taken in combination is —(C═O)— such that there are zero or one —(C═O)— groups;
R_{C} is independently selected at each occurrence of R_{C} from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;
X is a linking group comprising a backbone chain having 1 to about 8 atoms, the backbone chain can optionally include ester, amide, ether or thioether linkages in the backbone chain; and
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl, wherein at least one of R₁ or R₂ is a substituted alkyl or alkyloxy group;
n is either 2 or 3 and is independently chosen at each occurrence of n; and
at least one occurrence of R_{A} or R_{C} in Formula I is chosen to be —XNR₁R₂,
where the metal complex resulting from the binding of the compound to the metal ion is either neutral or cationic.

2-4. (canceled)

5. A compound capable of binding a metal ion, the compound according to the formula: 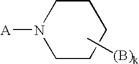
wherein
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;
B is independently selected at each occurrence of B from the group consisting of optionally substituted alkyl, optionally substituted alkenyl, optionally substituted alkynyl, hydroxy, optionally substituted alkoxy, halogen, hydroxy, optionally substituted alkoxyalkyl, optionally substituted amino, optionally substituted mono and dialkyl amino, optionally substituted aryl, optionally substituted aralkyl, optionally substituted cycloalkyl, optionally substituted heteroalicyclic, optionally substituted heteroaralkyl, optionally substituted heteroaryl, and —X—Y;
X is a linking group comprising a backbone chain having 1 to about 8 atoms, the backbone chain can optionally include ester, amide, ether or thioether linkages in the backbone chain;
k is an integer from about 1 to about 3; and
Y is a group capable of chelating to at least one metal ion,
wherein at least one of A or B is chosen to be —X—Y.

6-10. (canceled)

11. The compound of claim 5, wherein Y is a group of the formula: 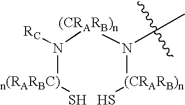
wherein:
R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;
R_{B} is hydrogen or lower alkyl having from about 1 to about 6 carbon atoms for each occurrence of R_{B}; or
—(CR_{A}R_{B})— taken in combination is —(C═O)— such that there are zero or one —(C═O)— groups;
R_{C} is selected from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;
X is a linking group comprising a backbone chain having 1 to about 8 atoms, the backbone chain can optionally include ester, amide, ether or thioether linkages in the backbone chain; and
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, optionally substituted heteroaryl;
n is either 2 or 3 and is independently chosen at each occurrence of n.

12. The compound of claim 11, wherein the group Y is selected from groups according to the formula: 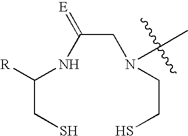
wherein
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms.

13. The compound of claim 5, wherein X is selected from the group consisting of —(CH₂)ₘ—C(O)NH— and α,ω-alkylene groups wherein the alkylene group has between about 1 and about 10 carbon atoms and between 0 and about 3 oxygen or sulfur atoms in the alkylene chain;
m is an integer of from about 1 to about 5.

14-15. (canceled)

16. A compound according to the formula: 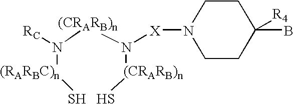
wherein:
B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;
R₄ is hydrogen, hydroxy, halogen, optionally substituted alkyl groups having from 1 to about 6 carbon atoms, optionally substituted alkoxy groups having from I to about 6 carbon atoms, or
R₄ and B taken in combination form an optionally substituted heterocyclic group having 5 or 12 ring atoms and one or two N, O, or S atoms and 1 or 2 fused rings;
R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;
R_{B} is hydrogen or lower alkyl having from 1 to about 4 carbon atoms for each occurrence of R_{B}; or
—(CR_{A}R_{B})— taken in combination is —(C═O)— such that there are zero or one —(C═O)— groups;
R_{C} is selected from the group consisting of hydrogen, lower alkyl groups having 1 to about 8 carbon atoms, alkoxyalkyl group having from 2 to about 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;
Y is a group capable of chelating to at least one metal ion;
X is a linking group comprising a backbone chain having 1 to about 8 atoms, the backbone chain can optionally include ester, amide, ether or thioether linkages in the backbone chain;
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and
n is either 2 or 3 and is independently chosen at each occurrence of n.

17. The compound of claim 16, the compound according to the formula: 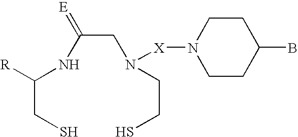
wherein:
B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;
Y is a group capable of chelating to at least one metal ion;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

18. A compound capable of binding a metal ion, the compound according to the formula: 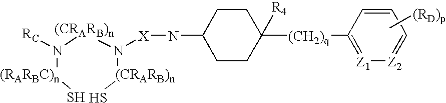
wherein:
R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, optionally substituted heteroalicyclic groups;
R₄ is hydrogen, hydroxy, halogen, optionally substituted alkyl groups having from 1 to about 6 carbon atoms, optionally substituted alkoxy groups having from 1 to about 6 carbon atoms, or
Z₁ and Z₂ are independently selected from CH, CR_{D}, and N;
p is selected from integers between about 0 and about 5;
q is selected from integers between about 0 and about 10;
R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;
R_{B} is hydrogen or lower alkyl having from about 1 to about 4 carbon atoms for each occurrence of R_{B}; or
—(CR_{A}R_{B})— taken in combination is —(C═O)— such that there are zero or one —(C═O)— groups;
R_{C} is selected from the group consisting of hydrogen, lower alkyl groups having 1 to about 8 carbon atoms, alkoxyalkyl groups having from 2 to 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;
Y is a group capable of chelating to at least one metal ion;
X is a linking group comprising a backbone chain having 1 to about 8 atoms, the backbone chain can optionally include ester, amide, ether or thioether linkages in the backbone chain;
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and
n is either 2 or 3 and is independently chosen at each occurrence of n.

19. The compound of claim 18, the compound according to the formula: 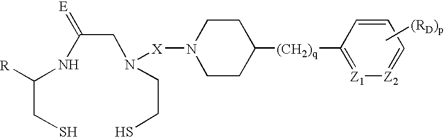
wherein:
R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, and optionally substituted heteroalicyclic groups;
Z, and Z₂ are independently selected from CH, CR_{D}, and N;
p is selected from integers between about 0 and about 5;
q is selected from integers between about 0 and about 10;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

20. A compound capable of binding a metal ion, the compound according to the formula: 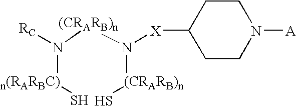
wherein:
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;
R_{A} is independently chosen at each occurrence of R_{A} from the group consisting of hydrogen, lower alkyl having 1 to about 4 carbon atoms, alkyl ester groups having about 2 to about 8 carbon atoms, aryl ester groups having about 7 to about 18 carbon atoms, alkyl amide groups having about 2 to about 8 carbon atoms, aryl amide groups having about 7 to about 18 carbon atoms, di(alkyl)aminoalkyl groups where each alkyl group has 1 to about 4 carbon atoms, and —XNR₁R₂;
R_{B} is hydrogen or lower alkyl having from about 1 to about 4 carbon atoms for each occurrence of R_{B}; or
—(CR_{A}R_{B})— taken in combination is —(C═O)— such that there are zero or one —(C═O)— groups;
R_{C} is selected from the group consisting of hydrogen, lower alkyl groups having 1 to about 8 carbon atoms, alkoxyalkyl groups having from 2 to 8 carbon atoms, alkyl ester or aryl ester groups having about 2 to about 8 carbon atoms, alkyl amide or aryl amide groups having about 2 to 8 carbon atoms, di(alkyl)aminoalkyl groups where each alkyl group has 1 to about 4 carbon atoms, and —XNR₁R₂;
Y is a group capable of chelating to at least one metal ion;
X is a linking group comprising a backbone chain having 1 to about 8 atoms, the backbone chain can optionally include ester, amide, ether or thioether linkages in the backbone chain;
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl; and
n is either 2 or 3 and is independently chosen at each occurrence of n.

21. The compound of claim 20, the compound according to the formula: 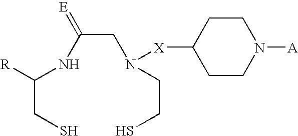
wherein:
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

22-23. (canceled)

24. A neutral or cationic complex comprising a metal ion and a compound according to the fonnula: 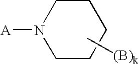
wherein
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y:
B is independently selected at each occurrence of B from the group consisting of optionally substituted alkyl, optionally substituted alkenyl, optionally substituted alkynyl, hydroxy, optionally substituted alkoxy, halogen, hydroxy optionally substituted alkoxyalkyl, optionally substituted amino, optionally substituted mono and dialkyl amino, optionally substituted aryl, optionally substituted aralkyl, optionally substituted cycloalkyl, optionally substituted heteroalicyclic, optionally substituted heteroaralkyl, optionally substituted heteroaryl, and —X—Y;
X is a linking group comprisinv a backbone chain having 1 to about 8 atoms, the backbone chain can optionally include ester, amide, ether or thioether linkages in the backbone chain:
k is an integer from about 1 to about 3: and
Y is a group capable of chelating to at least one metal ion,
wherein at least one of A or B is chosen to be —X—Y.

25-28. (canceled)

29. The complex of claim 24, wherein the complex is of the formula: 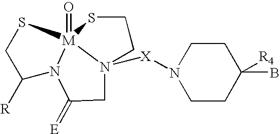
wherein
M is one or more isotopes of technetium or rhenium;
B is selected from the group consisting of optionally substituted alkyl, optionally substituted alkenyl, optionally substituted alkynyl, hydroxy, optionally substituted alkoxy, optionally substituted alkoxyalkyl, optionally substituted amino, optionally substituted mono and dialkyl amino, halogen, optionally substituted aryl, optionally substituted aralkyl, optionally substituted cycloalkyl, optionally substituted heteroalicyclic, optionally substituted heteroaralkyl, optionally substituted heteroaryl, and —X—Y;
R₄ is hydrogen, hydroxy, halogen, optionally substituted alkyl groups having from 1 to about 6 carbon atoms, optionally substituted alkoxy groups having from 1 to about 6 carbon atoms, or
R₄ and B taken in combination form an optionally substituted heterocyclic group having 5 or 12 ring atoms and one or two N, O, or S atoms and 1 or 2 fused rings;
Y is a group capable of chelating to at least one metal ion;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

30. A complex of claim 29, wherein the complex is of the formula: 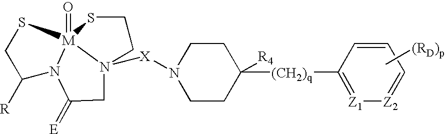
wherein:
M is one or more isotopes of technetium or rhenium;
R_{D} is independently selected at each occurrence from the group consisting of optionally substituted alkyl, optionally substituted alkenyl, optionally substituted alkynyl, hydroxy, amino, halogen, cyano, nitro, optionally substituted alkoxy, optionally substituted alkoxyalkyl, optionally substituted mono and dialkyl amino, optionally substituted aryl, optionally substituted heteroaryl, optionally substituted cycloalkyl, and optionally substituted heteroalicyclic groups;
R₄ is hydrogen, hydroxy, halogen, optionally substituted alkyl groups having from 1 to about 6 carbon atoms, optionally substituted alkoxy groups having from 1 to about 6 carbon atoms;
Z₁ and Z₂ are independently selected from CH, CR_{D}, and N;
p is selected from integers between about 0 and about 5;
q is selected from integers between about 0 and about 10;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

31. A complex of claim 29, wherein the complex is of the formula: 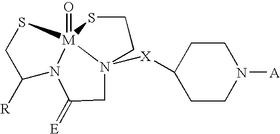
wherein:
M is one or more isotopes of technetium or rhenium;
A is selected from the group consisting of optionally substituted alkyl, optionally substituted alkenyl, optionally substituted alkynyl, optionally substituted aryl, optionally substituted aralkyl, optionally substituted cycloalkyl, optionally substituted heteroalicyclic, optionally substituted heteroaralkyl, optionally substituted heteroaryl, and —X—Y;
R is selected from hydrogen, C(O)O(R₃), or C(O)NH(R₃);
R₃ represents hydrogen, optionally substituted alkyl, optionally substituted alkenyl, optionally substituted alkynyl, optionally substituted aryl, optionally substituted aralkyl, and optionally substituted cycloalkyl;
E represents an oxo group or two hydrogen atoms; and
X is a linking group comprising a backbone chain having 1 to about 8 atoms, the backbone chain can optionally include ester, amide, ether or thioether linkages in the backbone chain.

32. The complex of claim 24, wherein the metal ion is radiolabelled or radioactive.

33-35. (canceled)

36. A method for in-vivo or in-vitro imaging of at least one tumor comprising the steps of:
providing a radiolabled metal complex of any one of claims 29 through 31, wherein;
contacting the tumor(s) with the radiolabeled metal complex; and
making a radioagraphic image to visualize the tumor(s).

37-39. (canceled)

40. The method of claim 36, wherein the tumor(s) are neoplasm(s).

41. The method of claim 36, wherein the tumor(s) are carcinoma(s).

42. The method of claim 36, wherein the tumor(s) are melanoma(s).

43. The method of claim 36, wherein the tumor(s) are prostate carcinoma, breast carcinoma, lung carcinoma, renal carcinoma, colon carcinoma, glioblastoma, neuroblastoma, sarcoma, or a combination thereof.

44-45. (canceled)

46. A method for in-vivo or in-vitro imaging of at least one tissue expressing one or more proteins or receptors for which radiolabeled complexes have affinity, the method comprising the steps of:
providing a radiolabeled metal complex of claim 24;
contacting the tissue(s) expressing the receptors with the radiolabeled metal complex; and
making a radiographic image to visualize the tissue(s).

47. The method of claim 46, wherein the proteins or receptors selected from serotonin receptors, adrenergic receptors, adrenoceptors receptors, dopamine receptors, sigma receptors, emopamil binding proteins, calcium channel receptors, or any subtype or subclass thereof.

48. The method of claim 46, wherein the protein or receptor expressed by the tissue to be imaged are selected from 5HT_{1A}, σ₁, σ₂, α₁, Ca+2 channel receptors, EBP or a combination thereof.

49-53. (canceled)

54. A method for the treatment of cancer comprising the steps of:
providing a cytotoxic metal complex according to claim 24; and
contacting the tumor(s) with the cytotoxic metal complex.

55-62. (canceled)

63. A method of inhibiting a protein or receptor comprising the steps of:
providing a metal complex according to claim 24; and
contacting the tumor(s) with the metal complex.

64. The method of claim 63, the protein or receptor are selected from serotonin receptors, adrenergic receptors, adrenoceptors receptors, dopamine receptors, sigma receptors, emopamil binding proteins, calcium channel receptors, or any subtype or subclass thereof.

65. The method of claim 63, wherein the neuroreceptor(s) are selected from 5HT_{1A}, σ₁, σ₂, α₁, Ca²⁺ channel receptors, EBP or a combination thereof.

66-68. (canceled)

69. A compound capable of binding a metal ion, the compound according to the formula: 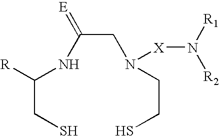
wherein
X is a linking group comprising a backbone chain having 1 to about 8 atoms, the backbone chain can optionally include ester, amide, ether or thioether linkages in the backbone chain; and
R₁ and R₂ each are independently selected unsubstituted alkyl groups having from 1 to about 8 carbon atoms, alkoxyalkyl group having from 2 to about 8 carbon atoms, and substituted alkyl or alkoxyalkyl groups having from 1 to about 8 carbon atoms which are substituted with one or more groups selected from optionally substituted aryl, optionally substituted cycloalkyl, optionally substituted heteroalicyclic, and optionally substituted heteroaryl, wherein at least one of R₁ or R₂ is a substituted alkyl or alkyloxy group;
R is selected from hydrogen, C(O)O(R₃), or C(O)NH(R₃); and
E represents an oxo group or two hydrogen atoms.
